SEQUENCE LISTING (4 sequences)

SEQ ID NO: 1 - protein, 734 aa, synthetic construct
  source 1..734: note = wild type AAV2 VP1 capsid protein
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRN

SEQ ID NO: 2 - DNA, 21 bp, synthetic construct
  source 1..21: mol_type = other DNA
taggggcccacatcttccaac

SEQ ID NO: 3 - DNA, 24 bp, synthetic construct
  source 1..24: mol_type = other DNA
gttcttcggacgcctcgtcaacac

SEQ ID NO: 4 - DNA, 27 bp, synthetic construct
  source 1..27: mol_type = other DNA
gtgacaaagagccacacaacagtgacc

(19) United States
(12) Patent Application Publication
(10) Pub. No.: US 2024/0124893 A1
(43) Pub. Date: Apr. 18, 2024

(54) Methods of Treating Human X-Linked Retinoschisis Using Gene Therapy

(71) Applicant: TEAMEDON INTERNATIONAL, INC., Rockville, MD (US)

(72) Inventor: Xueyan Peter Mu, Rockville, MD (US)

(21) Appl. No.: 18/486,941

(22) Filed: Oct. 13, 2023

Related U.S. Application Data
(63) Continuation-in-part of application No. 18/161,655, filed on Jan. 30, 2023.
Application No. 18/161,655 is a continuation-in-part of application No. PCT/US2022/079195, filed on Nov. 3, 2022.
(60) Provisional application No. 63/275,351, filed on Nov. 3, 2021.

(51) Int. Cl. C12N 15/86; A61K 9/00; A61K 38/17; A61P 27/02

(52) U.S. Cl. CPC C12N 15/86; A61K 9/0048; A61K 38/1709; A61P 27/02; C12N 2750/14143; C12N 2750/14171

(57) ABSTRACT

A method of treating X-linked juvenile retinoschisis (XLRS) in a human subject includes subretinally delivering to the human subject a therapeutically effective amount of an rAAV vector. The rAAV vector includes a nucleic acid sequence comprising coding sequence for human RS1 protein. The rAAV vector can further include a mutated AAV2 VP3 capsid protein having a phenylalanine (F) for tyrosine (Y) substitution at each of the positions corresponding to Y444, Y500 and Y730 in a wild type AAV2 VP3 capsid protein.

### INCORPORATION OF SEQUENCE LISTING

This application includes a Sequence Listing which is being submitted in XML format, named “TEAM002CIP.xml”, which is 6 KB in size and created on Oct. 13, 2023. The contents of the Sequence Listing are incorporated herein by reference in their entirety.

### BACKGROUND

X-linked juvenile retinoschisis (XLRS) is a recessive degenerative disease of the central retina affecting only males with a worldwide prevalence estimated at 1/5000-1/25,000. XLRS is caused by mutations in the gene that encodes a protein called retinoschisin (RS1) (1-3), which directs production of a cell-surface adhesion protein by the retina's photoreceptor and bipolar cells (4,5). The protein has two conserved sequence motifs, an initial signal sequence targets the protein for secretion and the larger discoidin domain is implicated in cell adhesion. RS1 helps to maintain the structural organization of the retinal cell layers and promotes visual signal transduction. Patients with XLRS experience splitting primarily in the inner nuclear layer of the macula, and in the periphery in about 50% of patients (6,7). This results in abnormal communication between the photoreceptors and bipolar cells, resulting in a reduced b-wave, and often an electronegative electroretinogram (ERG), defined as a b-wave below the baseline (4,8,9).

Affected individuals have a relatively normal a-wave in the electroretinogram (ERG), while the b-wave is nearly or totally absent. Another hallmark of XLRS is the localized splitting of the central retina, which develops primarily in the fovea, but can also be present in the peripheral retina. These cystoid cavities may coalesce, leading to further visual acuity loss.

There is no specific treatment for XLRS therefore this represents an important unmet medical need. Anecdotal reports suggest that topical carbonic anhydrase inhibitors may provide some reduction in degree of schisis detected by OCT and improvement in visual acuity in some but not all patients. To mitigate macular schisis, various approaches have been employed, including the utilization of topical and oral carbonic anhydrase inhibitors (10). Research conducted on mice lacking Rs1 function has demonstrated that the use of recombinant adeno-associated virus (rAAV) gene therapy vectors expressing functional RS1 can lead to substantial improvements in both retinal structure and function (11-13) including photoreceptor preservation, a decrease in the number and size of schisis cavities, and a recovery of the ERG b-wave response (5, 11, 12, 14, 15-17).

No products have been approved by regulatory agencies for treatment of this condition. Thus far, treatment of XLRS has been limited to the prescription of low-vision aids. Surgical interventions benefit the patient only in rare cases.

Recombinant AAV (rAAV) vectors have been developed by deleting the viral rep and cap genes, inserting a transgene expression cassette between the ITRs, and packaging the vector DNA into AAV capsids in a packaging cell. rAAV vectors are uniquely suitable for in vivo gene therapy because they are non-toxic, highly efficient at transducing a wide variety of non-dividing cell types, and persist for long periods, primarily in episomal form, resulting in long-term expression of the transgene. rAAV vectors have been effective for treatment of a wide variety of animal models of genetic diseases, including retinal diseases, hemophilia, muscular dystrophy, lysosomal storage disorders and diseases of the central nervous system.

Mice deficient in retinoschisin have been developed and used to obtain insight into the role of retinoschisin in retinal structure, function, and pathology. Studies in these murine models of XLRS have shown that recombinant adeno-associated virus (rAAV) gene therapy vectors expressing normal RS1 can provide significant restoration of retinal structure and function in RS1-deficient mice.

Previous clinical trials using gene therapy by AAV-RS1 gene vector to treat XLRS patients using intravitreal injection route were not successful. A phase I/IIa clinical trial to assess the safety and tolerability of ocular gene therapy using an AAV8-RS/vector, administered intravitreally, was performed with three different doses (1×10⁹, 1×10¹⁰ and 1×10¹¹ vg/eye) (18). Another phase I/II dose-escalation study to assess safety and potential efficacy in patients with XLRS utilized a capsid modified vector, rAAV2-tYF-CB-hRS1, intravitreally (1×10¹¹, 3×10¹⁰, and 6×10¹¹ vg/eye). Both studies showed that high dose intravitreal injection causes ocular inflammation (18,19). The observed ocular inflammation limited substantial improvement in visual function outcomes. The lack of significant improvement underscores the challenges and complexities involved in developing effective treatments for XLRS using gene therapy approaches. These findings emphasize the need for further research and refinement of therapeutic strategies and the relationship between route of administration, dosing, and efficacy to address the underlying mechanisms and specific limitations associated with XLRS.

The transfection efficiency of AAV is influenced by various stages, including vector binding to receptors on the cell surface (20). Studies have demonstrated that phosphorylation of tyrosine residues on the capsid surface of AAV2 by epidermal growth factor receptor protein tyrosine kinase can trigger ubiquitination and subsequent degradation of viral particles (21). To mitigate this degradation process, researchers have employed site-directed mutagenesis, specifically changing tyrosine to phenylalanine (Y-F), on some of the seven capsid surface exposed tyrosine residues in the VP3 common region of AAV2. This modification has been reported to protect vector particles from proteasome degradation, resulting in significant improvements in transfection efficiency compared to the wild-type AAV2 vector. These enhanced transfection capabilities have been observed both in tissue culture experiments and animal models (22). Subretinal injection of AAV vectors for gene therapy may have advantages over intravitreal injection in terms of the humoral immune response. Some authors suggest that intravitreal injections are more prone to inducing the production of neutralizing antibodies compared to subretinal injections. These antibodies have the potential to impede the successful transfer of genes during gene therapy (23,24). Although intravitreal administration offers the potential for panretinal transfection without the difficulties of subretinal surgery, especially attractive in eyes with XLRS in which bullous schisis cavities with holes in inner and/or outer retinal layers may be present, there is growing evidence indicating that intravitreal injections carry a higher risk of humoral immune responses compared to subretinal (23).

This lack of efficacy may be due to the dilution of the administered vector in the vitreous humor. There remains a need to find an effective therapy to treat XLRS patients.

### SUMMARY

The present disclosure provides a method of treating X-linked juvenile retinoschisis (XLRS) in a human subject by subretinally delivering to the human subject a therapeutically effective amount of an rAAV vector, the rAAV vector comprising a nucleic acid sequence comprising a coding sequence for human RS1 protein. The rAAV vector can further include a mutated AAV2 VP3 capsid protein comprising phenylalanine (F) for tyrosine (Y) substitutions at each of the positions corresponding to Y444, Y500 and Y730 in a wild type AAV2 VP3 capsid protein. The nucleic acid sequence of the rAAV vector can also further include a CMV enhancer sequence, and/or chicken beta actin promoter sequence. In specific embodiments, the rAAV vector is rAAV2tYF-CB-hRS1 as described herein.

### BRIEF DESCRIPTION OF THE DRAWINGS

FIG. 1 shows the organization of the elements in the DNA of the rAAV2tYF-CB-hRS1 vector.

FIG. 2 shows epifluorescence IHC image of an animal that has been intravitreally injected with AAV2tYF-CB-GFP.

FIG. 3 shows epifluorescence image of an animal that has been subretinally injected with AAV2tYF-CB-GFP.

FIG. 4 shows schematically the expression cassette of the rAAV2tYF-CB-hRS1 vector used in Example 2. The vector contains AAV serotype 2 inverted terminal repeats and an expression cassette consisting of a cytomegalovirus enhancer and chicken b-actin promoter, human RS1 complementary DNA, and a simian vacuolating virus 40 polyadenylation sequence and is packaged in an AAV2 capsid containing tyrosine to-phenylalanine mutations in 3 surface-exposed tyrosine residues (Y444F, Y500F, Y730f) in the VP3 protein capsid (AAV2tYF).

FIGS. 5A-5F show characterization of Retinoschisis Disease in Retinoschisin Knockout Mice: Rod and Cone Photoreceptors Functional Loss and Cyst Formation. FIG. 5A: OCT images of a Rs1-KO mouse (1) and a wild-type control littermate (2) are shown at 1 month of age. The outer nuclear layers (ONLs) are indicated by a white solid bar, cysts are indicated by white stars on the OCT image. FIG. 5B: Quantification of ONL thickness between Rs1-KO and WT controls at 1 month of age. Rs1-KO has thinner ONL. FIG. 5C: Quantification of the cyst severity scores between Rs1-KO and WT controls. Cyst severity score is modified from Bush et al. (30) where 1, no cavities; 2, <30 μm; 3, 30-49 μm; 4, 50-69 μm; 5, 70-99 μm; 6, ≥100 μm. Rs1-KO have cysts while WT do not. FIG. 5D: Rod function on electroretinogram after dark adaptation using a 0.01 cd·sec/m² dim flash at 1 month of age. Representative waveform comparison between Rs1-KO and WT controls demonstrates (1, upper panel) Hyper-normal a-wave amplitudes in Rs1-KO mouse model compared to the WT controls (arrow), which is significant (2, lower panel). FIG. 5E: Rod and Cone function on electroretinogram after dark adaptation using 3.0 cd·sec/m² reveals standard combined response waveforms in Rs1-KO and WT controls (1, upper panel) Both a- and b-waves are lower amplitude in Rs1-KO mice. (2, lower panel) b/a wave ratio is reduced in Rs1-KO compared to WT controls. FIG. 5F: Cone function on ERG measured after light adaptation using 3.0 flash (1, upper panel) and 5 Hz flicker (2, lower panel). Amplitudes are reduced in Rs1-KO compared to WT controls. MO: month, *: p<0.01, **: p<0.001, ****: p<0.0001.

FIGS. 6A-6F show AAV2tYF subretinal gene therapy as described in Example 2 improves and sustains cone photoreceptor function in Rs1-KO mice treated with the medium dose of 8E8 vg/eye over the course of 7 months and comparable to the WT controls. FIG. 6A: Cone function, assessed through light-adapted (LA) electroretinogram, revealed that when exposed to a 3.0 cd·s/m² bright flash, eyes treated with the 8E8 dose consistently displayed significantly greater b-wave amplitudes compared to sham treated eyes and untreated eyes at 1-3 MPI, and this trend continued up to the 7 MPI time point. FIG. 6B: Comparisons of representative LA waveforms were made among eyes treated with different doses, diluent-treated eyes, and untreated eyes at 3 MPI (left panel) and 7 MPI (right panel), respectively. FIG. 6C: Another metric used to evaluate cone function was the 5 Hz flicker test, which consistently showed that 8E8-treated eyes outperformed untreated eyes throughout the treatment duration. FIG. 6D: When subjected to a 5 Hz flicker, cones in treated eyes elicited higher amplitudes at 3 months (left panel), which was not observed in the sham treated eyes or the untreated contralateral eyes. Over the course of 7 months (right panel), treated eyes retained significantly better amplitudes in cone ERGs. FIG. 6E: Cone function comparison between the medium 8E8 dose and WT control mice showing non significance at earlier and later time points tested by the 5 Hz flicker test, meaning 8E8 treatment restored cone function to WT levels. FIG. 6F: Waveform comparison between the medium 8E8 dose and the WT (left panel, 2 months; right panel 6 months).

FIGS. 7A-7I show that Subretinal gene therapy of Rs1-KO as described in Example 2 preserves significant rod and cone functions over the course of 4 months. FIG. 7A: Combined rod-cone function was measured by 3.0 cd·s/m² bright flash under dark-adapted conditions, at 1,2 and 3 MPI. The 8E8 dose treated eyes had higher b-wave amplitudes compared to the diluent injected eyes and the untreated contralateral eyes. FIGS. 7B and 7C: Representative waveforms of the 3.0 cd·s/m² bright flash SCR ERG test were made between all vector doses, diluent-injected eyes, and untreated eyes at 3 MPI and 7 MPI, respectively. The 8E8 dose had higher amplitudes at 3MPI and 7MPI. Waveforms from the same mice were used at each time point. FIG. 7D: SCR a-wave amplitudes showed normalization due to significant reduction in eyes treated with 8E9 (the highest dose) compared to the untreated controls. FIG. 7E: An elevated b/a ratio was consistently observed in the 8E8 dose-treated eyes until 5 MPI time point, attributable to the increased b-wave amplitudes. FIG. 7F: Rod function was measured by 0.01 cd·s/m² dim flash after dark-adaptation (DA). Notably, at 1-, 2-, and 3 MPI, the vector-treated eyes demonstrated a normalization of the previously observed hyper-normal a-wave seen in the Rs1-KO mouse model (as shown in FIG. 2). FIGS. 7G: and 7H: Waveform comparisons of the 0.01 cd·s/m² dim flash test were made between all vector doses, diluent-injected eyes, and untreated eyes at 3 MPI and 7 MPI, respectively. FIG. 7L B-wave amplitudes of the 0.01 cd·s/m² dim flash after dark-adaptation (DA) showing the significantly increased b-wave amplitudes of the 8E8 dose compared to the diluent treated and untreated eyes.

FIGS. 8A-8C show OCT quantification of test animals in Example 2. FIG. 8A: OCT images from 1 month post (MPI) treatment to 7 MPI looking at the cyst reduction and the ONL thickness of all the doses treated compared to sham treated eyes and untreated eyes. Each row of images represents the same mouse; each row is labeled with the gene therapy dose, the diluent or untreated status. FIG. 8B: Comparison of cyst severity between eyes treated with different doses, diluent treated eyes and untreated contralateral counterparts. Cyst severity was scored on a scale from 1 (least severe, no schisis) to 6 (most severe, schisis size >100 micrometers) from 1 MPI to 7 MPI. The results illustrate that eyes treated with the vector demonstrated a more substantial reduction in cyst severity compared to eyes treated with the diluent. Specifically, cysts in vector-treated eyes exhibited a greater reduction in severity over the study period when compared to their untreated contralateral eyes. FIG. 8C: Comparison of ONL thickness between all vector doses treated eyes, sham treated and untreated contralateral eyes from 1 month post treatment time point to 7 MPI, with no significant difference between all treated and untreated eyes. *: p<0.01, **: p<0.001, ****: p<0.0001

FIGS. 9A-9B show that medium dose (8E8) of subretinal AAV2tYF gene therapy as described in Example 2 has a trend toward improvement in functional vision. FIG. 9A: Under normal fluorescent ceiling light, Rs1-KO mice treated with 8E8 dosage of the AAV2tYF subretinal gene therapy took less time to locate the platform compared to untreated Rs1-KO mice and sham treated mice at both time points. FIG. 9B: In dark testing conditions (dim red lighting), mice treated with the 8E8 dose of the gene therapy took less time to locate the platform compared to untreated Rs1-KO mice and sham treated mice at both time points.

### DETAILED DESCRIPTION

Illustrative embodiments of the invention are described below. In the interest of clarity, not all features of an actual implementation are described in this specification. it should be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

In accordance with the present invention various genetic-modifying entities may be used individually or in combination to achieve the desired results. These entities may include naked natural or modified nucleic acid or peptides. Such modifications may contain carbohydrates including fatty acids and/or sugars. As used herein, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, nucleotides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of clarity related to the present invention, terms are defined below:
A, an: In accordance with long standing patent law convention, the words “a” and “an” when used in this application, including the claims, denotes “one or more.”
Expression: The combination of intracellular processes, including transcription and translation undergone by a polynucleotide such as a structural gene to synthesize the encoded peptide or polypeptide.
Promoter: a term used to generally describe the region or regions of a nucleic acid sequence that regulates transcription.
Regulatory Element: a term used to generally describe the region or regions of a nucleic acid sequence that regulates transcription. Exemplary regulatory elements include, but are not limited to, enhancers, post-transcriptional elements, transcriptional control sequences, and such like.
Structural gene: A polynucleotide, such as a gene, that is expressed to produce an encoded peptide, polypeptide, protein, ribozyme, catalytic RNA molecule, siRNA, or antisense molecule.
Transformation: A process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.
Transformed cell: A host cell whose nucleic acid complement has been altered by the introduction of one or more exogenous polynucleotides into that cell.
Transgenic cell: Any cell derived or regenerated from a transformed cell or derived from a transgenic cell, or from the progeny or offspring of any generation of such a transformed host cell.
Vector: A nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compostions that do not produce an allergic or similar untoward reaction when administered to a human, and in particular, when administered to the human eye. The preparation of an aqueous composition that contains a protein as an active ingredient is web understood in the art. Typically, such compositions are prepared as injectables, either as liquid solutions or suspensions; solid forms suitable for solution in, or suspension in, liquid prior to injection can also be prepared. The preparation can also be emulsified.

As used herein, the term “operatively linked” means that a promoter is connected to a functional RNA in such a way that the transcription of that functional RNA is controlled and regulated by that promoter. Means for operatively linking a promoter to a functional RNA are well known in the art.

### Adeno-Associated Viral Vectors:

The present invention provides methods of administering viral vectors comprising nucleic acids encoding human RS1 protein.

Adeno-associated virus (AAV) is a small (25-nm), nonenveloped virus that packages a linear single-stranded DNA genome of 4.7 Kb. The small size of the AAV genome and concerns about potential effects of Rep on the expression of cellular genes led to the construction of AAV vectors that do not encode Rep and that lack the cis-active IEE, which is required for frequent site-specific integration. The ITRs are kept because they are the cis signals required for packaging. Thus, current recombinant AAV (rAAV) vectors persist primarily as extrachromosomal elements.

A variety of recombinant adeno-associated viral vectors (rAAV) may be used to deliver genes of interest to a cell and to effect the expression of a gene of interest, e.g., a gene encoding hRS1. in a target cell. At times herein, “transgene” is used to refer to a polynucleotide encoding a polypeptide of interest, wherein the polynucleotide is encapsidated in a viral vector (e.g., rAAV).

Adeno-associated viruses are small, single-stranded DNA viruses, which require helper virus to facilitate efficient. replication. The 4.7-kb genome. of AAV is characterized by two inverted terminal repeats (ITR) and two open reading frames, which encode the Rep proteins and Cap proteins, respectively. The Rep reading frame encodes four proteins of molecular weight 78 kDa, 68 kDa, 52 kDa, and 40 kDa. These proteins function mainly in regulating AAV replication, and rescue and integration of the AAV into a host cell's chromosomes. The Cap reading frame encodes three structural proteins of molecular weight 85 kDa (VP1), 72 kDa (VP2), and 61 kDa (VP3) (Berns), which form the virion capsid. More than 80% of total proteins in AAV virion comprise VP3.

The genome of rAAV is generally comprised of: (1) a 5′ adeno-associated virus ITR, (2) a coding sequence (e.g., transgene) for the desired gene product (e.g., hRS1 protein) operatively linked to a sequence that regulates its expression in a cell (e.g., a promoter sequence such as a. inGluR6 or fragment thereof), and (3) a 3′ adeno-associated virus inverted terminal repeat. 0.1n addition, the rAAV vector may preferably contain a polyadenylation sequence.

Generally, rAAV vectors have one copy of the AAV ITR at each end of the transgene or gene of interest, in order to allow replication, packaging, and efficient integration into cell chromosomes. The ITR consists of nucleotides 1 to 145 at the 5′-end of the AAV DNA genome, and nucleotides 4681 to 4536 (i.e., the same sequence) at the 3′-end of the AAV DNA genome. The rAAV vector may also include at least 10 nucleotides following the end of the ITR (i.e., a portion of the “D region”).

The transgene sequence (e.g., the polynucleotide encoding hRS1) can be of about 2- to 5-kb in length or longer or shorter lengths of bases (or alternatively, the transgene may additionally contain a “stiffer” or “filler” sequence to bring the total size of the nucleic acid sequence between the two ITRs to between 2 and 5 kb). Alternatively, the transgene may be composed of repeated copies of the same or similar heterologous sequence several times, or several different heterologous sequences.

Recombinant AAV vectors of the present invention may be generated from a variety of adeno-associated viruses, including for example, any of serotypes 1 through 12, as described herein. For example, ITRs from any AAV serotype are expected to have similar structures and functions with regard to replication, integration, incision and transcriptional mechanisms.

In some embodiments, a cell-type specific promoter (or other regulatory sequence such as an enhancer) is employed to drive expression of a gene of interest, Representative examples of suitable promoters in this regard include a CBA promoter (chicken β-actin), CMV promoter, RSV promoter, SV40 promoter, MoMLV promoter, or derivatives, mutants and/or fragments thereof. Promoters and other regulatory sequences are further described herein.

Other promoters that may similarly be utilized within the context of the present invention include cell or tissue specific promoters (e.g., a rod, cone, or ganglia derived promoter), or inducible promoters. Representative examples of suitable inducible promoters include inducible promoters sensitive to an antibiotic, e.g., tetracycline-responsive promoters such as “tet-on” and/or “tet-off” promoters. Inducible promoters May also include promoters sensitive to chemicals other than antibiotics.

The rAAV vector may also contain additional sequences, for example from an adenovirus, which assist in effecting a desired function for the vector, Such sequences include, for example, those that assist in packaging the rAAV vector into virus particles.

Packaging cell lines suitable for producing adeno-associated viral vectors may be accomplished given available techniques (see e.g., U.S. Pat. No. 5,872,005). Methods for constructing and packaging rAA7I vectors are described in, for example, PCT Intl. Pat. Appl. Publ, No. WO 00/54813.

Flanking the rep and cap open reading frames at the 5′ and 3′ ends are 145-bp inverted terminal repeats (ITRs), the first 125 bp of which are capable of forming Y- or T-shaped duplex structures. The two ITRs are the only cis elements essential for AAV replication, rescue, packaging and integration of the AAV genome. There are two conformations of AAV ITRs called “flip” and “flop,” These differences in conformation originated from the replication model of adeno-associated virus, which uses the ITR to initiate and reinitiate the replication (R. O. Snyder et al., J. Viral., 67:6096-6104: 1993; K. I. Berns, Microbiol. Rev., 54:316-329; 1990). The entire rep and cap domains can be excised and replaced with a therapeutic or reporter transgene.

In some embodiments, self-complementary AAV vectors are used. Self-complementary vectors have been developed to circumvent rate-limiting second-strand synthesis in single-stranded AAV vector genomes and to facilitate robust transgene expression at a minimal dose. In specific embodiments, a self-complementary AAV of any serotype or hybrid serotype or mutant serotype, or mutant hybrid serotype increases expression of hRS1 and variants thereof by at least 10%, at least 15%, at least 20%, at east 25%, at least 30%, at least 35%, at east 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, or more than 200%, when compared to a non-self-complementary rAAV of the sane serotype,

In one aspect, the present disclosure provides methods of using an Adeno-associated Virus Vector Expressing Retinoschisin in treating Patients with X-linked Retinoschisis, or AAV-RS1. In some embodiments, the AAV is of a serotype selected from the group consisting of AAV1, AAV2, AAV3, AAV4, AAV5, AAV6, AAV7, AAV8, AAV9, AAV10, AAV11, AAV12, and hybrids thereof. In other specific embodiments, the AAV is recombinant AAV of a combinatorial hybrid of 2, 3, 4, 5, 6, 7, 8, 9, 10, 11, 12, 13, 14, 15 or more serotypes or mutants thereof. In some embodiments, the AAV vector is encapsulated in an AAV capsid protein which comprises at least one mutated tyrosine residue. The mutated tyrosine residue can be selected from the group consisting of Y252F, Y272F, Y444F, Y500F, Y700F, Y704F, Y730F, Y275F, Y281F, Y508F, Y576F, Y612G, Y673F, and Y720F. In a specific embodiment, the mutated capsid protein comprises one or more tyrosine residues, each mutated to a phenylalanine residue.

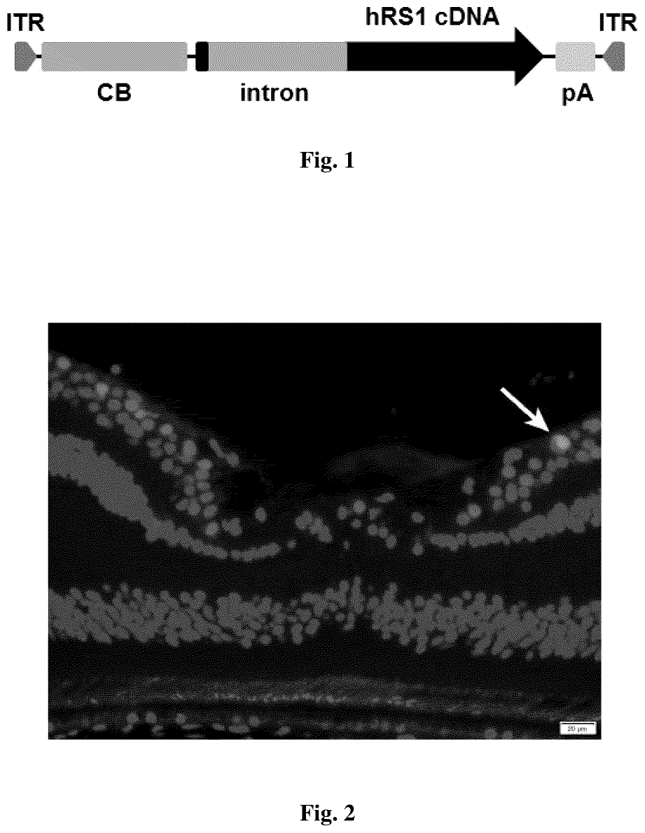

As an example, rAAV2tYF-CB-hRS1 is a replication-incompetent, recombinant adeno-associated virus (rAAV) vector that expresses the retinoschisin (RS1) protein after the vector enters retinal cells. rAAV2tYF-CB-hRS1 cDNA encodes the human retinoschisin (RS1) protein. The vector contains AAV serotype 2 inverted terminal repeats and an expression cassette consisting of a CMV enhancer, chicken beta actin promoter, the human RS1 cDNA and an SV40 polyadenylation sequence. The organization of the elements in the DNA of the rAAV2tYF-CB-hRS1 vector is depicted in FIG. 1, where ITR denotes inverted terminal repeat, CB denotes CMV enhancer and chicken beta actin promoter, RS1 denotes retinoschisin, pA denotes a polyadenylation sequence. The vector DNA is packaged in an AAV2 capsid referred to as AAV2tYF which contains tyrosine to phenylalanine (YF) mutations in three tyrosine residues at each of the positions corresponding to Y444, Y500 and Y730 (VP1 numbering) in a wild type AAV2 VP3 common region of the capsid protein. The sequence of the wild type AAV2 VP1 capsid protein is provided below:

(SEQ ID NO: 1)
MAADGYLPDWLEDTLSEGIRQWWKLKPGPPPPKPAERHKDD
SRGLVLPGYKYLGPFNGLDKGEPVNEADAAALEHDKAYDRQ
LDSGDNPYLKYNHADAEFQERLKEDTSFGGNLGRAVFQAKK
RVLEPLGLVEEPVKTAPGKKRPVEHSPVEPDSSSGTGKAGQQ
PARKRLNFGQTGDADSVPDPQPLGQPPAAPSGLGTNTMATGS
GAPMADNNEGADGVGNSSGNWHCDSTWMGDRVITTSTRTW
ALPTYNNHLYKQISSQSGASNDNHYFGYSTPWGYFDFNRFHC
HFSPRDWQRLINNNWGFRPKRLNFKLFNIQVKEVTQNDGTTT
IANNLTSTVQVFTDSEYQLPYVLGSAHQGCLPPFPADVFMVP
QYGYLTLNNGSQAVGRSSFYCLEYFPSQMLRTGNNFTFSYTF
EDVPFHSSYAHSQSLDRLMNPLIDQYLYYLSRTNTPSGTTTQS
RLQFSQAGASDIRDQSRNWLPGPCYRQQRVSKTSADNNNSE
YSWTGATKYHLNGRDSLVNPGPAMASHKDDEEKFFPQSGVL
IFGKQGSEKTNVDIEKVMITDEEEIRTTNPVATEQYGSVSTNL
QRGNRQAATADVNTQGVLPGMVWQDRDVYLQGPIWAKIPH
TDGHFHPSPLMGGFGLKHPPPQILIKNTPVPANPSTTFSAAKF
ASFITQYSTGQVSVEIEWELQKENSKRWNPEIQYTSNYNKSV
NVDFTVDTNGVYSEPRPIGTRYLTRN

The rAAV2tYF-CB-hRS1 vector can be produced using a recombinant herpes simplex virus complementation system in suspension-cultured baby hamster kidney cells. See Ye G J, Budzynski E, Sonnentag P, et al. Safety and biodistribution evaluation in cynomolgus macaques of rAAV2tYF-CB-hRS1, a recombinant adeno-associated virus vector expressing retinoschisin. Hum Gene Ther Clin Dev 2015; 3:165-176. Details are provided in the Thomas D L, Wang L, Niamke J, et al. Scalable recombinant adeno-associated virus production using recombinant herpes simplex virus type 1 coinfection of suspension-adapted mammalian cells. Hum Gene Ther 2009; 20:861-870. The disclosures of these references are incorporated by reference in their entireties.

For example, two rHSV helper viruses, one containing the AAV2 rep and AAV2tYF cap genes and the other containing the hRS1 expression cassette, can be used to coinfect sBHK cells grown in serum-free medium. The cells can then be lysed with Triton X-100 detergent and treated with Benzonase. Cell lysate containing the AAV vector can be clarified by filtration and purified by AVB Sepharose (GE Life Sciences) affinity chromatography followed by CIM SO3⁻ (BIA Separations) cation-exchange chromatography, and eluted in concentrated balanced salt solution containing 0.014% (v/v) Tween-20 (BSST). The purified bulk can then be concentrated and buffered exchanged to 1×BSST (drug substance) and sterile (0.2 μm) filtered to generate drug product. The vector can be further concentrated, as needed, using a 100 kDa MWCO Ultra centrifugal filter unit (EMD Millipore), and re-filtered (0.2 μm) to generate drug product sublots of specific concentrations. It can be stored frozen and thawed and diluted to the appropriate concentration immediately before administration.

### Pharmaceutical Compositions

Gene delivery vectors can be prepared as a pharmaceutically acceptable composition suitable for administration. In general, such pharmaceutical compositions comprise an amount of a gene delivery vector suitable for delivery of protein-encoding polynucleotide to a cell of the eye for expression of a therapeutically effective amount of the hRS1 protein, combined with a pharmaceutically acceptable carrier or excipient. Preferably, the pharmaceutically acceptable carrier is suitable for intraocular administration. Exemplary pharmaceutically acceptable carriers include, but are not necessarily limited to, saline or a buffered saline solution (e.g., phosphate-buffered saline).

Various pharmaceutically acceptable excipients are well known in the art. As used herein, “pharmaceutically-acceptable excipient” includes any material, which, when combined with an active ingredient of a composition, allows the ingredient to retain biological activity, preferably without causing disruptive reactions with the subject's immune system or adversely affecting the tissues surrounding the site of administration (e.g., within the eye).

Exemplary pharmaceutically carriers include sterile aqueous of non-aqueous solutions, suspensions, and emulsions. Examples include, but are not limited to, any of the standard pharmaceutical excipients such as a saline, buffered saline (e.g., phosphate buffered saline), water, emulsions such as oil/water emulsion, and various types of wetting agents.

Examples of non-aqueous solvents are propylene glycol, polyethylene glycol, hyaluronic acid, vegetable oils such as olive oil, and injectable organic esters such as ethyl oleate.

Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles can include fluid and nutrient replenishers, electrolyte replenishers (such as those based on Ringer's dextrose), and the like.

A composition of gene delivery vector of the invention may also be lyophilized using means well known in the art, for subsequent reconstitution and use according to the invention. Where the vector is to be delivered without being encapsulated in a viral particle (e.g., as “naked” polynucleotide), formulations for liposomal delivery, and formulations comprising microencapsulated polynucleotides, may also be of interest.

Compositions comprising excipients are formulated by well-known conventional methods (see, for example, Remington's Pharmaceutical Sciences, Chapter 43, 14th Ed., Mack Publishing Co., Easton, Pa., USA).

In general, the pharmaceutical compositions can be prepared in various forms, preferably a form compatible with intraocular administration. Stabilizing agents, wetting and emulsifying agents, salts for varying the osmotic pressure or buffers for securing an adequate pH value may also optionally be present in the pharmaceutical composition.

The amount of gene delivery vector in the pharmaceutical formulations can vary widely, i.e., from less than about 0.1%, usually at or at least about 2% to as much as 20% to 50% or more by weight, and may be selected primarily by fluid volumes, viscosities, etc., in accordance with the particular mode of administration selected.

### Subretinal Administration

A human subject suffering from XLRS can be treated by the compositions of the present application, rAAV2tYF-CB-hRS1, by introducing the composition into an affected eye of the subject by subretinal administration. The doses of rAAV2tYF-CB-hRS1 can be delivered using a subretinal injection cannula, e.g., with a polyamide micro tip with an inner diameter of 41 gauge. The site of the injection can be identified preoperatively to avoid schisis. On the day of surgery, the injection and resulting bleb can be monitored with intraoperative optical coherence tomography. The site of injection should avoid direct contact with the retinal vasculature or with areas of pathologic features. The entry point along the superior arcade and quality of subretinal injection can be identified under surgical microscope. An example of subretinal injection device is the MedOne MicroDose Injection Device (a 1 mL syringe with adapter and a PolyTip Cannula 25/38 g).

Further, a kit including the compositions of the present application, e.g., rAAV2tYF-CB-hRS1, as well as a delivery device (such as a syringe or cannula described herein), with instructions for use of the kit for subretinal administration to patients provided in physical manual and/or digital files stored on computer readable medium (e.g., CD-ROM, flash drives, etc.). The composition can be pre-loaded in the delivery device, or stored in a separate container from the delivery device.

### Example 1

A transduction efficiency study was conducted in normal nonhuman primates using a same vector as in rAAV2tYF-CB-hRS1 but containing a green fluorescent marker protein (GFP) coding sequence (rAAV2tYF-CB-GFP) via intravitreal or subretinal injection. Ten female cynomolgus monkeys were assigned to two groups (4 or 6 females/group), and a dose of 1×10¹¹ μg/eye were administered via intravitreal injection or subretinal injection. Animals were dosed once on Study Day 1. After dosing, animals were observed post-dose for approximately 12 weeks to assess GFP expression. The tolerability and transduction efficiency were also assessed. The study results shown that green fluorescent protein (GFP) fluorescence in intravitreal eyes was mainly limited to some retinal ganglion cells (RGCs) surrounding the fovea and their axons (FIG. 2: Epifluorescence IHC image of animal that had been intravitreally injected with AAV2tYF-CB-GFP. The red/green cone outer segments are labeled red. A single ganglion cell (arrow) shows green staining in its cytoplasm for GFP. Bar=20 μm).

Subretinal administration resulted in marked GFP fluorescence in photoreceptor cells (rods and cones) located over the subretinal delivery site (FIG. 3: Epifluorescence image of animal in the superior periphery at the location of the subretinal bleb. The inset is of the same area at higher magnification taken with a confocal microscope. It is apparent that the cones are more strongly labeled than the rods. The cones are wider than the rods and their nuclei are located near the external limiting membrane. Also, many of their outer segments are labeled red—the reaction product from the anti-red/green cone IHC. Bar=50 μm). Administration of test article via both delivery routes was found to associate with ocular inflammation, which was more severe in intravitreal eyes. The results from this study suggest that subretinal injection of more efficient in transducing photoreceptor cells and causes less ocular inflammation comparing to intravitreal injection.

### Example 2: A Dose Response Study of Subretinal rAAV2tYF-CB-hRS1 Treatment in RS1-KO Mice

Purpose: X-linked Retinoschisis (XLRS), due to loss-of-function mutations in the retinoschisin (RS1) gene, is characterized by a modest to severe decrease in visual acuity. Clinical trials for XLRS utilizing intravitreal gene therapy showed ocular inflammation. We conducted a subretinal dose-response study using rAAV2tYF-CB-hRS1 in a pre-clinical study utilizing the Rs1 knockout (Rs1-KO) mouse to investigate short- and long-term retinal rescue after subretinal gene delivery.

Methods: Rs1-KO mice were subretinally injected with 2 μL of rAAV2tYF-CB-hRS1 vector with 8E9 viral genomes (vg)/eye, 8E8 vg/eye, 8E7 vg/eye, or diluent alone as a sham injection, and compared to untreated eyes. Analysis of retinal function by electroretinography (ERG) and structural analysis by cyst severity score and outer nuclear layer thickness using optical coherence tomography (OCT) were performed at 1-2,3,5 and 7 months post injection (MPI). Functional vision was evaluated using a visually guided swim assay (VGSA).

Results: ERG demonstrated dose-dependent effects: 8E8 dose treated eyes had higher ERG amplitudes in light adapted bright flash and 5 Hz flicker protocol compared to untreated controls (p<0.0001) and sham treated eyes (p<0.0001) until the 7 MPI endpoint, consistent with improved cone photoreceptor cell function. ERG b-wave amplitudes were higher in response to dark adapted dim flash and standard combined response compared to sham-treated (p<0.01) and untreated eyes (p<0.001) which persisted until 3 MPI, suggesting short term improvement of the rod photoreceptors. All subretinal injections resulted in a cyst severity score of 1 (no cavities), with significant reductions compared to untreated eyes up to 3 MPI (p<0.05). All doses showed a trend towards greater cyst reduction than the diluent. The high and low dose groups showed inconsistent retinal function improvement, despite reduced cyst severity scores, emphasizing the dose-dependent nature of gene augmentation's efficacy and the tenuous connection between cyst reduction and ERG improvement in XLRS. VGSA showed a trend toward better functional vision in 8E8 dose treated mice.

Conclusion: Our dose-response data suggests that a dose of 8E8 vg/eye subretinally improves retinal function and structure in the Rs1-KO mouse. It improves and sustains cone photoreceptor electrical function, improves rod function, and reduces cyst severity scores. Subretinal diluent injection resolves schisis cysts, but 8E8 vg/eye is needed for optimal retinal electrical function rescue. These findings offer a promising path for clinical translation to human trials.

### Materials and Methods

### Animal Husbandry and Ethics Statement

This study followed the guidelines set forth in the National Institutes of Health's Guide for the Care and Use of Laboratory Animals. All animal handling procedures were performed in strict accordance with the approved Institutional Animal Care and Use Committee (IACUC) protocol #1041421 of the University of Iowa. The Rs1-KO mouse model was acquired from the Paul A. Sieving lab. The Rs1-KO mouse model was generated through homologous recombination in 129Sv/Ev mouse embryonic stem cells using a targeting construct, where a 17.5-kb mouse genomic DNA fragment containing exon 1 and most of intron 1 of the mouse Rs1 gene was replaced with a neomycin resistance (neoR) gene cassette, resulting in the knockout of the Rs1 gene. The characterization of this mouse model, developed with the assistance of Ingenious Targeting Laboratory, Inc. (Stony Brook, NY), has been described in detail elsewhere (14). Animals were housed according to IACUC recommendations. Animals were generated by crossing Rs1 males with either Rs1^{-/-} or Rs1^{-/-} females. Methods of euthanasia used were carbon dioxide inhalation followed by cervical dislocation. Humane endpoints were strictly observed, and every effort was made to minimize suffering.

### Genotyping

Genotyping of Rs1-KO mice was performed using Taq polymerase (M0273S, New England BioLabs) using the primers listed in Table 1 following the manufacturer's instructions. The PCR products were measured by agarose gel electrophoresis (E-gel Invitrogen by Thermo Fisher Scientific, California, USA). The 10 μL PCR reaction included 2 μL of betaine. The cycling conditions consist of 5 minutes of initial denaturing step followed by 35 cycles of denaturing step at 94° C. (30 seconds), the annealing step at 57° C. (30 seconds), extension at 72° C. (30 seconds), and then 4 minutes of final extension at 72° C. The size for the wild-type band in the PCR product is 516 base pair (bp), and for the knockout band is 300 bp. Primers are listed in Table 1.

### AAV Packaging and Subretinal Injection

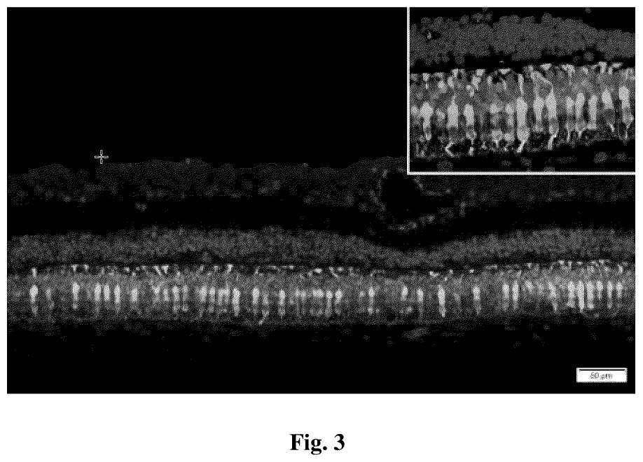

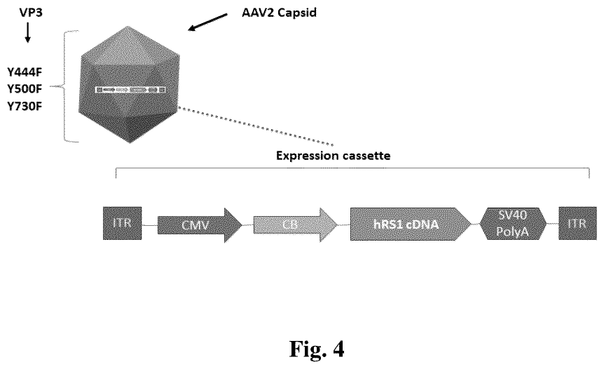

rAAV2tYF-CB-hRS1 was manufactured in compliance with current Good Manufacturing Practices by a contract manufacturer under the direction of Applied Genetic Technologies Corporation (19). The structure of the expression cassette of the rAA2tYF-CB-hRS1 vector is shown in FIG. 4. The vector contains AAV serotype 2 inverted terminal repeats and an expression cassette consisting of a cytomegalovirus enhancer and chicken beta actin promoter, human RS1 complementary DNA, and a simian vacuolating virus 40 polyadenylation sequence and is packaged in an AAV2 capsid containing tyrosine to-phenylalanine mutations in three surface-exposed tyrosine residues (AAV2tYF) (22). This Rs1-KO expression cassette is shown in FIG. 4.

For subretinal injection, mice were anesthetized with a ketamine/xylazine mixture (87.5 mg/kg ketamine, 12.5 mg/kg xylazine) via intraperitoneal route at 0.1 ml per 20 g body. Subretinal injections were performed with a 32-gauge Hamilton syringe under a Zeiss OPMI f 170 surgical microscope as described previously (25-27). For rAAV2tYF-CB-hRS1, two μL of virus at 4×10⁹ or 4×10⁸ or 4×10⁷ concentrations were injected into the temporal subretinal space of the mice eyes. The dilution buffer was Alcon BSS+tween. The Alcon BSS is: BSS® Sterile Irrigating Solution is a sterile balanced salt solution, each mL containing sodium chloride (NaCl) 0.64%, potassium chloride (KCl) 0.075%, calcium chloride dihydrate (CaCl₂·2H2O) 0.048%, magnesium chloride hexahydrate (MgCl₂·6H2O) 0.03%, sodium acetate trihydrate (C₂H₃NaO₂·3H2O) 0.39%, sodium citrate dihydrate (C₆H₅Na₃O₇·2H2O) 0.17%, sodium hydroxide and/or hydrochloric acid (to adjust pH), and water for injection. The pH is approximately 7.5. The osmolality is approximately 300 mOsm/Kg. Subretinal treatment alternated between the right (OD) and left (OS) eyes for different cohorts of animals, and the contralateral eyes served as untreated controls.

### Study Design

Rs1-KO animals between postnatal day (P) 23-P31 received subretinal injections of rAAV2tYF-CB-hRS1. In these treated animals, one eye received the subretinal gene therapy treatment, and the contralateral eye served as the untreated control. To evaluate the effect of the subretinal injection procedure on the retina, additional sham injections delivering the diluent buffer alone were performed. Completely untreated Rs1-KO mice were also included. Both male (hemizygous mutant) and female (homozygous mutant) mice were used in this study. Outcome measures include ERG, optical coherence tomography (OCT), and Visually Guided Swim Assay (VGSA). ERG and OCT were performed at 1, 2-, 3-, 5-, and 7 MPI, to evaluate retinal function and photoreceptor survival over time. VGSA was performed at two different time points (4-5 months of age and 9 months of age). Number of eyes enrolled in the dose response study is shown in Table 2.

### Exclusion Criteria

The subretinal injection procedure involves temporarily separating the retina from the Retinal Pigment Epithelium (RPE) to create a subretinal bleb. The success of the subretinal injection can be determined by the presence and size of the subretinal bleb immediately after the injection. At the time of the subretinal injections, the size of the retinal blebs was visually assessed for each animal under the Zeiss OPMI f 170 surgical microscope. Rarely, the subretinal injection causes a large vitreous hemorrhage, or a chronic retinal detachment detectable on OCT at 1 MPI. These eyes were excluded. A specific exclusion criterion was set for the VGSA: in rare cases, some mice displayed a lack of motivation to swim and instead repeatedly floated in the pool during the experiment. To ensure data quality, any data from a mouse (both light and dark testing) were excluded if its mean swim time deviated by more than 1 standard deviation from the group's median due to consistent floating behavior (floating more than three times per test episode or requiring more than three interventions to stop floating, achieved either by snapping fingers to create an audible sound or by flicking the mouse's tail).

### Electroretinography (ERG)

ERG was obtained using the Celeris system from Diagnosys (Diagnosys LLC, MA, USA) 30 days after treatment initiation and again at 2,3,5, and 7 months after treatment. Before conducting the ERG procedure, the mice underwent overnight dark adaptation. Mice were intraperitoneally anesthetized using a mixture of ketamine and xylazine (87.5 mg/kg ketamine, 12.5 mg/kg xylazine) at a volume of 0.1 ml per 20 g body weight. The eyes were treated with a 1% tropicamide ophthalmic solution (Akron inc, Lake Forest, IL) 3 minutes prior to the ERG test. ERGs were recorded simultaneously from the corneal surface of each eye. Before placing the electrodes, GONAK Hypromellose ophthalmic demulcent solution (NDC 17478-064, Akorn) was applied to the eyes. Body temperature was maintained at a constant temperature of 38° C. using the system heat pad. Dim red light was used for illumination until dark-adapted testing was completed. ERG a-wave and b-wave data were collated in Microsoft Excel and analyzed using GraphPad Prism software (GraphPad Software Inc. San Diego, CA). To assess rod function, dim flashes of 0.01 cd·sec/m² were delivered under dark-adapted conditions. For combined rod-cone function assessment, bright flashes of 3.0 cd·sec/m² were administered while the mice remained in a dark-adapted state. To evaluate cone function, the mice were light-adapted for 10 minutes to bleach the rods in the retina. Then, two different measures were used to isolate the functions of cones: 3.0 cd·sec/m² single flashes, and a train of flickering lights at 5 Hz. These standardized ERG testing protocols are adapted for mice from the guidelines established by the International Society for Clinical Electrophysiology of Vision (ISCEV) (28).

### Optical Coherence Tomography (OCT)

OCT was performed using the spectral domain (SD) Envisu Image Guided SD-OCT system. Bioptigen InVivoVue software (Leica Biosystems Inc, Buffalo Grove, IL), incorporated within the OCT machine was used for quantification and allowed segmentation and thickness estimation for all retinal layers (26). Prior to the OCT procedure, mice were intraperitoneally anesthetized using a ketamine/xylazine mixture as described above, and their pupils were dilated with 1% tropicamide ophthalmic. The mice were then placed in the animal imaging mount—rodent alignment stage (AIM-RAS) setup for image acquisition (29). Axis manipulation before image capture was done to centrally align the optic nerve head (ONH) in the middle of the scans as a landmark. All images are a central OCT scan from temporal to nasal sides of the mouse. The quantification of the outer nuclear layer (ONL) thicknesses in both control and Rs1-KO mice, and the cyst severity score, were measured and averaged from 4 locations 500 μm equidistant from the center of ONH using the in-software calipers provided by Bioptigen. This cyst scoring system is based on the one reported by Bush et al. (30). Cyst severity scores were assigned based on the height of schisis cavities in the retinas (1, no cavities; 2, <30 μm; 3, 30-49 μm; 4, 50-69 μm; 5, 70-99 μm; 6, ≥100 μm) Intraperitoneal antisedan (atipamezole hydrochloride 5.0 mg/mL) 0.2 mL per 20 g body weight injection was given for reversal of anesthesia.

### Visually Guided Swim Assay

The visually guided swim assay, described in detail elsewhere, (31) was modified from the Morris water maze (32) and incorporated features of the mouse swim assay developed by Pang et al (33). Briefly, mice were placed in a plastic pool full of water containing a randomly placed platform. They were trained to know there would be a platform in the pool they could climb upon. The experiment was conducted under two different lighting conditions: normal room light (13.35 cd/m²) and dark with dim red lighting (approximately 4.17×10-3 cd/m²). The protocol involved 4 training days in the light, followed by 4 testing days in the light, then 2 training days in the dark, and finally 4 testing days in the dark. Each day, mice performed five swim trials, and the platform locations were the same for all mice in the group. A time limit of 60 seconds was set for each trial to prevent fatigue. If a mouse failed to reach the platform within 60 seconds, its time was recorded as 60 seconds for that particular trial.

### Statistical Analysis

Statistical analysis was performed with GraphPad PRISM 9.0. (GraphPad Software, Inc, San Diego, California, USA). For the ERG quantification and the OCT scans measurements, two-way ANOVA was used to analyze the difference between groups over time, followed by the multiple comparisons post hoc Tukey's test (non-parametric). The student's t-test was used in the comparison between control and Rs1-KO mice. Values shown are averages ±standard deviation.

TABLE 1
Primers for Rs1-KO genotyping

| Primer | Ratio | Sequence (5′ -> 3′) |
|---|---|---|
| Rs1-KO-WT-Fr | 33% | TAGGGGCCCACATCTTCCAAC (SEQ ID |
| | | NO: 2) |
| PLA2 (13) | 33% | GTTCTTCGGACGCCTCGTCAACAC (SEQ |
| | | ID NO: 3) |
| Rs1-KO-WT-Rv | 33% | GTGACAAAGAGCCACACAACAGTGACC |
| | | (SEQ ID NO: 4) |

TABLE 2
Number of eyes enrolled in the dose response study

| Number of eyes | Dose (vg/eye) | Study eye | Sex | Age (months) |
|---|---|---|---|---|
| 3 | 8E7 | OD | M | 1 |
| 4 | 8E7 | OS | M, F | 1 |
| 5 | 8E8 | OD | M, F | 1 |
| 2 | 8E8 | OS | M, F | 1 |
| 4 | 8E9 | OD | M, F | 1 |
| 3 | 8E9 | OS | M, F | 1 |
| 6 | Buffer diluent | OD | M | 1 |
| 9 | Untreated | OD | M, F | 1 |
| 18 | Untreated | OS | M, F | 1 |

### Results

### Phenotypic Characterization of Rs1-KO Mouse Model Reveals Key Features of XLRS Pathology

The Rs1-KO mouse model has been shown to be an invaluable tool for studying the involvement of the RS1 gene in a variety of physiological retina processes (4,5,11-13). Confirming and adding to previously reported phenotypic descriptions of the natural history and characteristics of the Rs1 knockout mouse model is crucial for elucidating the underlying mechanisms of XLRS and developing potential therapeutic interventions.

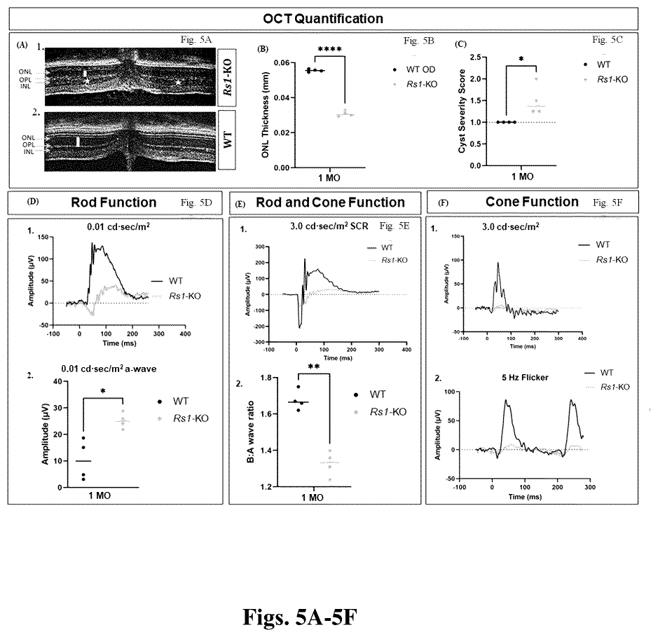

OCT: At the age of one month, the outer nuclear layer (ONL) thickness in Rs1-KO mice was 48% (FIG. 5 a1, b) as thick as the WT or heterozygous controls (FIG. 5 a2, b) suggesting that photoreceptor degeneration occurs at an early age in the Rs1-KO mouse model.

In addition to the reduced thickness of the outer nuclear layer (ONL), we quantified the schisis (cyst) severity in Rs1-KO mice at 1 month of age. Schisis was observed in the inner nuclear layer (FIG. 5 a1, c) in contrast to the absence of schisis in the wild type littermates (FIG. 5 a2, c), (p<0.05). ERG: An interesting phenomenon in the untreated Rs1-KO eyes is the hyper-normal a-wave amplitudes in the dark-adapted dim flash (0.01 cd·s/m²) ERG protocol. This was surprising given the fact that these eyes have only about half the number of photoreceptor cells compared to unaffected controls, and the a-wave is generated primarily by photoreceptor cells (34). These amplitudes were much larger than those observed in normal WT mice under the same test conditions with a statistical difference (p=0.0113). (FIG. 5 d1,2) The hyper-normal a-wave response, possibly due to disrupted synapses triggering a negative feedback mechanism, augmenting photoreceptor signals for enhanced retinal response, is a feature of the XLRS mouse model that has not been previously reported. Also, at 1 month of age, there is a reduction in the amplitudes of the b-wave of the 3.0 bright flash standard combined response ERG as shown by the representative waveform (FIG. 5 e1). Notably, b/a ratio is also reduced (FIG. 5 e2), mirroring the clinical features of RS1-associated retinopathy (26). Representative waveforms (FIG. 5f) showing a notable reduction in cone-dependent ERGs in 3.0 bright flash and the 5 Hz flicker ERG. These findings highlight the involvement of RS1 in maintaining the functionality of both rod and cone photoreceptors and their ability to generate robust electrical responses.

Subretinal Gene Therapy of Rs1-KO with 8E8 vg/Eye Rescues Cone Electrical Function Comparable to WT Controls

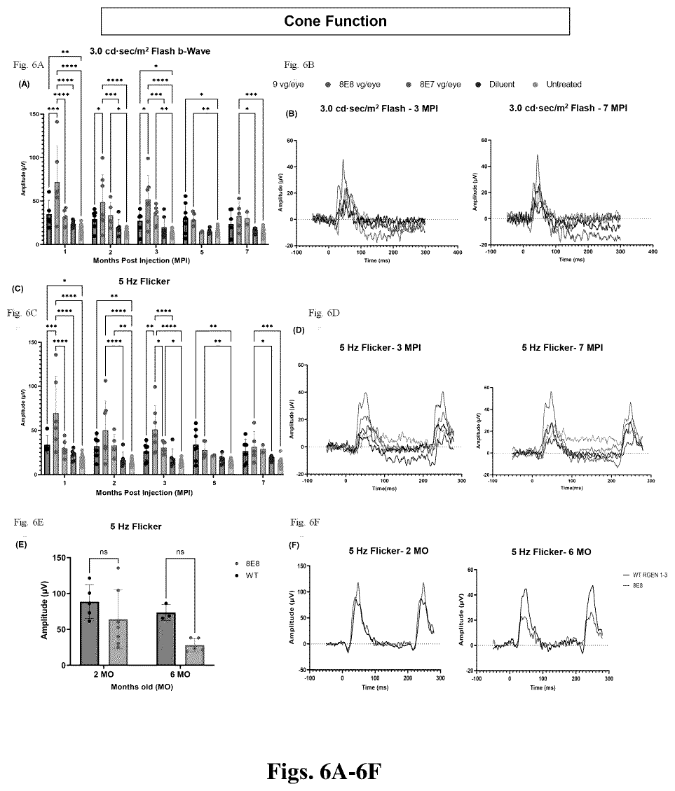

Different doses of rAAV2-tYF-CB-hRS1 viral vector were subretinally delivered to Rs1-KO mice between ages of P23-P31. Eyes that received 8E9, 8E8, 8E7 vg/eye of rAAV2-tYF-CB-hRS1 viral vector were compared to eyes that received diluent only, as well as completely uninjected eyes. In each animal, one eye was treated, and the contralateral eye was left untreated as an internal comparison. We previously showed in FIG. 5 that the ERGs in Rs1-KO mice are at very low amplitudes at 1 month of age. To assess whether subretinal gene therapy improves and maintains retinal function over time, ERG was performed at 1, 2, 3, 5, and 7 MPI to measure the function of cones and rods in treated and untreated eyes. Cones play a crucial role in daytime vision, color perception, and high-detail vision in humans. Light adapted 3.0 cd·sec/m² single flash, 5-Hz flicker ERG were performed to assess cone function in both treated and untreated Rs1-KO eyes. At 1 MPI, treated Rs1-KO eyes with the dose 8E8 vg/eye had a strong response to the 3.0 cd·sec/m² bright flash and had higher b-wave values (71.86±41.29 μV, n=6) compared to both sham-treated (23.85±3.98 μV, n=6, p<0.0001) and untreated eyes (16.43±5.13 μV, n=23, p<0.0001). This improvement was also observed at 2 and 3 MPI (FIG. 6a). This data shows that the dose 8E8 of AAV2tYF gene therapy restores the electrical function in cones.

Subsequently, we investigated whether the restoration of cone functions persisted in the 8E8 treated eyes. At the endpoint of the study (7 MPI), treated eyes had higher b-wave amplitudes compared to untreated eyes (FIG. 6a; 8E8 treated eyes: 32.73±16 μV, n=5; untreated eyes: 14.05±4.38 μV, n=18; p=0.0003) and compared to the sham injected eyes (FIG. 6a; 8E8 treated eyes: 32.73±16 μV, n=5; sham treated eyes: 16.46±2.72 μV, n=6; p=0.018). The Light adapted 3.0 cd·sec/m² single flash stimulus light elicited a high waveform in treated eyes, whereas untreated contralateral eyes and sham injected eyes displayed poor responses (FIG. 6b, 3 MPI and 7 MPI), suggesting that the regained cone electrical function was retained in vector gene therapy treated eyes (8E8 dose) over the course of the experiment. The light adapted 5-Hz flicker test, another metric for measuring cone electrical function in mice and more exclusively a cone function as the rods do not have time to recover between flashes, was conducted. At 1 MPI, the medium dose 8E8 group showed a significant improvement in the amplitudes of the 5-Hz flicker ERG compared to the sham treated eyes (p<0.0001) and the untreated eyes (p<0.0001) The improvement in cone function by the 5 Hz flicker test was not limited to the 1 MPI time point but was consistently observed at 2 MPI and 3 MPI (FIG. 6c). Treated eyes with 8E8 dose demonstrated a clear periodic waveform in response to the 5-Hz flicker (FIG. 6d). In contrast, the 5-Hz flicker stimuli had a very poor response in the sham treated eyes and the untreated eyes at any age (FIG. 6d). Therefore, gene delivery was able to reestablish the electrical function in the cone photoreceptor cells in the shorter term (3 MPI). At the experimental endpoint (7 MPI), 8E8 treated eyes had greater responses to the 5-Hz flicker stimuli compared to untreated eyes (8E8: 31.54±17.49 μV, n=5; untreated eyes: 13.30±4.79 μV, n=18; FIG. 6c), and compared to the sham injected eyes (FIG. 6c; 8E8 treated eyes: 31.54±17.49 μV, n=5; sham treated eyes: 17.87±2.57 μV, n=6; p=0.036). At 7 MPI, treated mice had a robust periodic waveform in their treated eyes, while very small waveforms were observed in the untreated contralateral eyes and the sham injected eyes. Therefore, gene augmentation with the dose of 8E8 vg/eye enabled cone photoreceptor cells to restore their electrical function on ERG. Remarkably, when comparing the medium 8E8 dosage to WT controls at two distinct ages, 2 months old (1 MPI) and 6 months old (5 MPI), no statistically significant differences were observed (2 MO, p=0.2933; 6 MO, p=0.095, FIG. 6e, f). This underscores the substantial impact of the medium 8E8 gene therapy dose in restoring cone function to the level seen in WT controls.

Surprisingly, although the high dose (8E9) and the low dose (8E7) groups had higher amplitudes compared to the untreated eyes, they did not show consistent improvement in the cone retinal function. (FIG. 6). Importantly, the isotonic buffer diluent injections demonstrated slightly better amplitudes compared to untreated eyes, aligning with findings from a prior study by our laboratory (35). However, while the buffer diluent treated eyes performed better than untreated eyes, efficacy did not reach the level observed with gene therapy, underscoring the importance of the administered dose in achieving consistent improvements. These observations from our experimental study strongly suggest that the rescue of retinal function through gene augmentation in XLRS is dependent on the administered dose.

Subretinal Gene Therapy with the Dose of 8E8 vg/Eye Slows the Loss of Rod and Cone Photoreceptor Functions in Treated Eyes

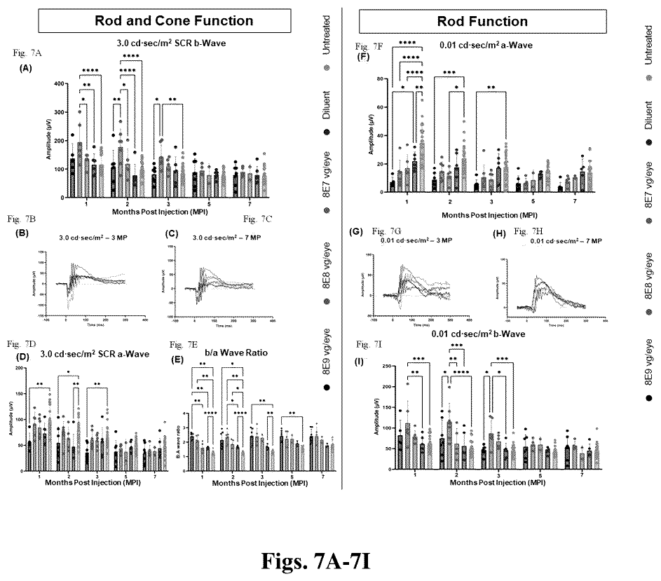

We measured the combined function of rod and cone photoreceptors using a standard combined response (SCR) ERG after exposing dark adapted eyes to 3.0 cd·sec/m² bright flashes. The b-wave amplitudes, representing bipolar cell responses driven by photoreceptor input, were significantly higher in the 8E8 dose-treated eyes compared to untreated eyes at 1 and 2 MPI (p<0.0001) and 3 MPI (p=0.0016) and compared to the diluent at 1 MPI (p=0.0023) and 2 MPI (p <0.0001) (FIG. 7a, b). This suggests that the treatment dose 8E8 delayed the loss of rod and cone photoreceptor function and improved bipolar cell activity. While the 8E8 dose showed a decreased treatment effect at 7 MPI compared to earlier time points, it still exhibited a superior waveform compared to untreated eyes. (FIG. 7c). The highest dose group (8E9) exhibited significantly reduced a-wave amplitudes in the SCR ERG at 1, 2 and 3 MPI. However, there was no statistical difference between the other gene therapy treated groups compared to the untreated controls (FIG. 7d). The SCR a-wave reflects primarily the combined rod and cone photoreceptor light response (36,37) and the b-wave reflects postsynaptic bipolar cell activity, albeit driven by photoreceptor input (38-39). The b/a ratio, known to be a useful marker in XLRS (40,41), demonstrated a progressive increase in the b/a ratio in the treated eyes due to the increase of the b-wave amplitudes and not the decrease in the a-wave amplitudes in the 8E8 dose compared to the untreated eyes (FIG. 7e). Eyes receiving the 8E8 dose had a normalized a-wave due to a 6.8% reduction in amplitude compared to untreated at 1 MPI while the b-wave amplitudes were increased 66.85% compared to the untreated eyes; the same trend was observed at most time points. This observation suggests enhanced signal transmission from the photoreceptors to the bipolar cells in the treated eyes with the 8E8 dose, indicating a positive response to the treatment at the cellular level. On the other hand, we observed an elevation in the b/a ratio within the 8E9 group at 1, 2, 3 and 5 MPI. Nonetheless, this increase in the ratio is attributed to the significant reduction percentage of the a-wave amplitudes at 1 MPI, which was 42.22% and not to the b-wave increase percentage which was 17.85% suggesting a lower beneficial effect of the highest dose on the b-wave in the SCR ERG.

Subretinal Gene Therapy of Rs1-KO with a Dose of 8E8 vg/Eye Preserves Rod Photoreceptor Function Short Term

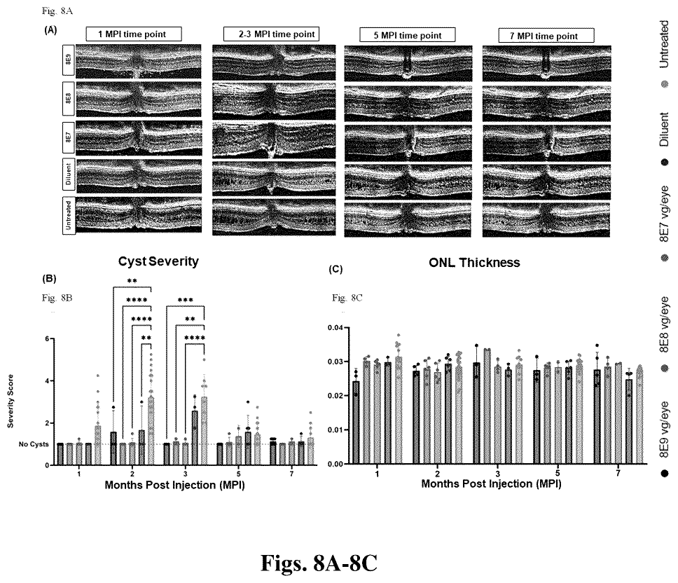

Rods are primarily responsible for vision in low-light environments. Rod function was evaluated by subjecting the eyes to a 0.01 cd·sec/m² dim flash after overnight dark adaptation. At 1 MPI, all treated eyes with all doses had significantly normalized a-wave amplitudes compared to the untreated eyes (p<0.0001) and the diluent (p=0.007) (FIG. 70, suggesting that the hyper-normal a-wave was due to an increased electrical activity in Rs1-KO eyes, and not due to differences in the number of rod photoreceptors. This observation was continued through most time points except for the diluent (FIGS. 8f-h). This indicates that the gene therapy using the AAV2tYF vector successfully corrected the hyper-normal a-wave amplitudes and brought them back to normal levels at most time points. These findings highlight the effectiveness of the AAV2tYF vector in normalizing a-wave amplitudes in XLRS in the 0.01 cd·sec/m² dim flash ERG test. Treated Rs1-KO eyes receiving the 8E8 dose showed a significant improvement in ERG b-wave amplitudes (111.83±54.36 μV, SD, n=7) compared to diluent injections (62.19±18.75 μV, SD, n=7; p=0.0197) and untreated eyes (62.91±20.58 μV, SD, n=23; p<0.0018) (FIG. 7i). These improvements were also observed at 2 and 3 MPI (FIG. 7i, g), and were not observed in eyes that received the 8E9 vg/eye dose, indicating that the middle 8E8 dose produced the best ERG outcome. The 8E8 dose effect was statistically significant over the first three MPI (p<0.0001, FIG. 7i). These findings suggest that gene therapy can enhance the function of existing rod photoreceptors in treated retinas. However, the gene therapy effect of the b-wave on the treated eyes stops at 5 MPI (FIG. 7i, g). The representative waveforms (depicted in FIG. 7g, h) demonstrate that among the various doses administered to mice, the medium dose of 8E8 was most effective.

Interestingly, although the high dose group (8E9 vg/eye) and the low dose group (8E7 vg/eye) showed better amplitudes than the untreated eyes over the course of the treatment, they did not show consistent improvement in the retinal function in the rod-specific pathway compared to the diluent (1 MPI, 8E9: p=0.36; 8E7: p=0.70), (7 MPI, 8E9: p=0.96; 8E7: p=0.99) or compared to the untreated eyes at all time points (1 MPI, 8E9: p=0.204; 8E7: p=0.98) (7 MPI, 8E9: p=0.36; 8E7: p=0.98). The observations suggest that the efficacy of gene augmentation in restoring the loss of function of rod photoreceptors in XLRS is most efficacious, though temporary, at the 8E8 dose.

### Subretinal Injection of Rs1-KO Reduces Cyst Severity

We conducted serial OCT comparison experiments at different time points (1, 2, 3, 5, and 7 MPI) to assess the impact of the rAAV2-tYF-CB-hRS1 viral vector and diluent injections on schisis (cyst) formation and the outer nuclear layer (ONL) thickness (FIG. 8a). At 1 MPI, retinal cysts were apparent in treatment naïve Rs1-KO eyes, but not in eyes that received the rAAV2-tYF-CB-hRS1 viral vector nor the diluent. (FIG. 8a, 1 MPI time point). At the 2 MPI treatment time point (3 months of age), cysts in uninjected eyes had worsened; treatment naïve eyes possessed a significantly higher cyst severity score compared to eyes that received the diluent or the rAAV2-tYF-CB-hRS1 viral vector (8E8 and 8E7 vg/eye: p<0.0001; 8E9: p=0.004; Diluent: p=0.008). It is worth noting that our group has previously reported the beneficial effects of injecting buffer/diluent alone on cyst severity in Rs1-KO mice (35, 36; GEhrke/Thompson et al. Frontiers in Medicine). At 3 MPI, eyes that received different doses of the rAAV2-tYF-CB-hRS1 viral vector had less severe cysts compared to eyes that received the diluent. At later time points, OCT images show that cysts naturally resolve over time even in treatment naïve eyes (FIG. 8a, b). This agrees with Zeng et al. who reported a significant reduction in cavity size between four and eight months in an Rs1-KO mouse model. (42)

To determine whether the injection of the rAAV2-tYF-CB-hRS1 viral vector had an impact on photoreceptor survival, the thickness of the ONL was measured. There were no significant differences in the ONL thickness between the Rs1-KO mice that received different doses of AAV2tYF, the diluent, and completely untreated eyes through their lives (1 MPI to 7 MPI) (FIG. 8a, c) with an average ONL thickness 0.025 mm (FIG. 8), The findings suggest that the AAV2tYF vector is well-tolerated by the mice, with no further photoreceptor degeneration or any signs of toxicity in the retinal cells following subretinal administration.

8E8 vg/Eye Dose of Subretinal Gene Therapy Showed a Trend Toward Better Functional Vision in Rs1-KO Treated Mice

To assess the effectiveness of gene therapy on functional vision, we conducted experiments using the visually guided swim assay (VGSA), a method that quantitatively measures rodent functional vision. This test evaluates the visual abilities of mice under various lighting conditions, taking into account both rod and cone-dependent visual pathways. The VGSA is analogous to the multi-luminance mobility test (MLMT) which provides quantitative data on mobility performance and was chosen as an endpoint for the human clinical trials of gene therapy with voretigene neparvovec (now Luxturna®) (43,44). In the VGSA, a mouse is placed in a pool and trained to identify and swim to a platform. The platform's location is randomized in each trial to prevent the mouse from memorizing its position. After 20 trials, the average time-to-platform (TTP) provides a numerical representation of its functional vision. VGSA approach helps us understand the impact of gene therapy treatment on improving visual function in mice and provides valuable insights for potential treatments in human vision-related conditions (31). We conducted testing under two different lighting conditions: normal room light measuring 13.35 cd/m² and dark adapted (DA) in dim red lighting measuring approximately 4.17×10⁻³ cd/m². Under the light condition, vision primarily relies on the cone pathway, while in low-light or dark conditions, the rod pathway predominantly supports vision. Wild type or heterozygous control mice have an average TTP of approximately 3-5 seconds in both light and dark conditions, and their average TTP remains stable over their lives; this data was previously reported (31). In contrast, we have shown previously that Rs1-KO mice have worse functional vision than normal controls at 4-6 months of age in the dark condition (31), and that this swim assay is sensitive enough to detect differences in the rates of visual decline in other inherited retinal diseases (27, 31, 45)

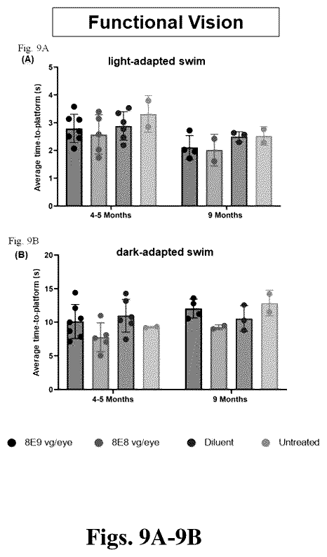

To determine which dose of the subretinal gene therapy (AAV2tYF) prevents the loss of vision in Rs1-KO mice, mice that received the 8E9, 8E8 doses, the diluent injected mice and untreated Rs1-KO mice were tested at 4-5 months of age (3-4 MPI) and then at 9 months of age (8 MPI) in both light and dark conditions). Untreated Rs1-KO mice had an average TTP of 3.32±0.66 s in the light between 4-5 months of age, which improved to 2.52±0.34 s at 9 months of age (FIG. 9a). Similar data was previously reported (31), showing that Rs1-KO mice aged 4-6 MO have a TTP similar to WT controls in bright light. This indicates that functional vision in bright light remained intact. On the other hand, 8E8 dose treated Rs1-KO mice had an average TTP of 2.57±0.7 s in the light between 4-5 months of age, and 2.0±0.5 s at 9 months of age, requiring 22.59% and 20.6% less time to complete the swim experiment than their untreated counterparts at these ages (FIG. 9a). The data obtained from our experiments indicate a discernible trend towards improvement in the mice treated with the 8E8 dose. Notably, the findings suggest that the mice subjected to RS1 gene therapy showed slightly superior TTP-compared to the untreated Rs1-KO mice under light-adapted conditions, specifically at 8 MPI.

To determine the effect of subretinal gene therapy on vision in low-light conditions, mediated by rods, the VGSA was performed in the dark, facilitated by dim red lighting which does not excite the rods. For untreated Rs1-KO mice, the average TTP in the dark was 9.26±0.11 s between 4-5 months of age, which worsened to 12.85±1.9 s—at 9 months of age, (FIG. 9b). Also, it was reported previously that a difference from controls appeared in the Rs1-KO DA TTP, indicating that there might be some impairment in their visual capabilities under dark conditions. On the other hand, for Rs1-KO mice that received subretinal gene therapy with the dose of 8E8, the average TTP in the dark was 7.74±2.1 s between 4-5 months of age, and 9.2±0.3 s at 9 months of age, requiring 16.4% and 28.4% less time than their untreated counterparts to find the platform at these ages (FIG. 9b). These results support the notion that subretinal gene therapy dose of 8E8 provided a benefit in preventing the loss of low-light vision over the XLRS disease course. The 8E8 dose performed better compared to the other administered doses (8E9) and the diluent injected mice, both under light-adapted and dark-adapted conditions. This highlights the efficacy of the 8E8 dose, establishing its prominence as the most effective treatment option in the experimental setup. The substantial advantage displayed by the 8E8 dose compared to its counterparts not only underscores its potential as a potent therapeutic agent but also reinforces its potential as a promising candidate for further investigation and clinical development. It is noteworthy that in mice with XLRS, their TTP improved over time, indicating that, similar to humans, mice might learn to adapt and utilize their impaired vision better with age.

### Discussion

### The Significance of Subretinal Gene Therapy in Contrast to Previous Intravitreal Injection Approaches

Two phase I/II dose-escalation human clinical trials (NCT02317887 and NCT02416622) for treating X-linked retinoschisis using AAV8-RS1 or AAV2tYF-CB-hRS1 gene therapy vector respectively with 3 different doses, using the intravitreal approach showed that increasing the dose seemed to correlate with increased inflammation. This inflammatory response wasn't fully eliminated by steroids, leading to recurrent or chronic inflammation (uveitis) in some cases (18, 19). The observations of gene-therapy-associated uveitis appear to be more frequent among studies using an intravitreal (IVT) route of delivery than studies using a subretinal approach (46). Preclinical studies have provided evidence that increases in dosage can lead to an increase in inflammation (5). In nonhuman primates, the IVT delivery of an AAV2tYF vector expressing green fluorescent protein, processed to enrich for AAV capsids containing the genome (full capsids) or capsids without the genome (empty capsids), demonstrated that ocular inflammation was primarily related to the total amount of capsid delivered and not to transgene expression (47). Although both clinical trials (18, 19) assessed the feasibility of IVT delivery of a gene vector in patients with XLRS, the trials failed to show efficacy of the treatment through the IVT. This provides motivation/rationale for investigating gene therapy efficacy using subretinal delivery.

### Strategic Selection of AAV2tYF-CB-hRS1 Vector for Enhanced Subretinal Gene Therapy

The choice of the AAV2tYF-CB-hRS1 vector was guided by its attributes aligning with successful gene transfer. Specifically, the enhanced retinal transduction capabilities of the adeno-associated virus serotype 2 with tyrosine-to-phenylalanine mutations tYF (AAV2tYF) were harnessed to effectively target retinal cell populations (5). By inducing mutagenesis in critical surface-exposed tyrosine residues on AAV2 capsids, alterations in transduction efficiency, kinetics, and penetration ability were achieved when delivering the virus to the retina (21). Moreover, the utilization of the chicken beta actin promoter was advantageous in driving robust and enduring gene expression within retinal cells (5), potentially extending therapeutic benefits. While regular single stranded AAV2 vectors effectively deliver genes to different types of retinal cells via subretinal or intravitreal approaches (26), they have limitations in infecting inner retinal cells. They mainly affect cells close to the injection area and usually take several weeks to achieve the highest level of gene expression. Notably, wild-type AAV2 vectors also face challenges such as susceptibility to antibody-mediated immunity against their capsids, potentially impeding transgene expression upon vector readministration (23). The AAV2tYF-CB-hRS1 vector's selection addresses these concerns by capitalizing on its enhanced transduction capabilities, mutagenesis potential to optimize efficiency, and utilization of a robust promoter for sustained expression, collectively presenting a promising avenue for effective gene transfer to specific retinal cell subsets.

Insights from Rs1-KO Mouse Model: Unveiling the Complexities of XLRS Pathogenesis and Therapeutic Prospects

We observed a novel phenomenon, a distinct hyper-normal a-wave response in untreated Rs1-KO mice, potentially signifying a novel characteristic of XLRS. This unusual phenomenon suggests that there are compensatory mechanisms at play in the retina. We hypothesize that the disruption of retinal cell organization and the photoreceptor-bipolar cell synaptic structure, attributed to the absence or dysfunction of retinoschisin, could trigger adaptive responses. In a normal retina, phototransduction causes photoreceptors to hyperpolarize in response to light, leading to a decrease in glutamate release onto ON bipolar cells. This, in turn, causes ON bipolar cells to depolarize, generating the b-wave of the ERG. The a-wave, which precedes the b-wave, represents the hyperpolarization of photoreceptor cells. (48) Studies have proposed that certain aspects of the ERG waveform, specifically the bright flash dark-adapted a-wave trough and its immediate recovery, are influenced by depolarizing currents in ON bipolar cells which are driven by cones presumably. It's suggested that the loss of depolarization in ON bipolar cells could potentially lead to an increased a-wave amplitude in the ERG. (48) We are hypothesizing that could happen in the 0.01 dim flash in Rs1-KO as the mutations in the RS1 gene could lead to abnormal or disrupted synapses between photoreceptor cells and bipolar cells. This disruption could reduce the efficiency of signal transmission from photoreceptors to bipolar cells which would trigger the retina to a negative feedback mechanism to enhance the available signals, which would involve a heightened sensitivity of cone cells to dim light conditions. This would effectively increase the contribution of cone-derived signals to bipolar cells and the downstream retinal layers. While the exact mechanism remains to be elucidated, these findings shed light on the intricate interplay between retinal cell function and synaptic communication. Additionally, deficits in both rod and cone photoreceptor-dependent functions emphasized the critical role of RS1 in sustaining robust electrical responses. The reduction in the b/a ratio further mirrored clinical aspects of RS1. By investigating subretinal gene therapy intervention during the emergence of disease features, we shed light on potential therapeutic strategies. Our study underscores the importance of RS1 in maintaining retinal health and reinforces the relevance of the Rs1-KO mouse model for exploring XLRS pathogenesis and therapeutic interventions.

### Exploring Optimal Dosage for Subretinal Gene Therapy Efficacy

The dose dependent effect was tested in other gene therapy studies for treating XLRS (18,43). Finding the optimal dose for treating XLRS is critical as previous high doses of AAV2tYF vector caused inflammation (18). The intriguing dose-response relationship observed in our study provides valuable insights into the optimal therapeutic window for subretinal gene therapy. The second highest dose (8E8 vg/eye) demonstrated superior effects over both lower and higher doses hinting at the importance of achieving a balance between gene expression and potential off-target effects. This phenomenon may be attributed to achieving an optimal range of gene expression that enhances functional improvements while minimizing the risk of unintended cellular responses.

Integrated Approach Reveals Retinal Health: Insights from ERG, OCT and VGSA Analyses

Over 279 pathogenic variants of the RS1 gene have been linked to XLRS (49). Gene therapy for RS1 holds significant promise to enhance the lives of these patients. Our study aimed to assess the effectiveness of subretinal gene therapy using AAV2tYF-CB-hRS1 vector across different doses (8E9, 8E8, 8E7 vg/eye) in Rs1-KO mice and compared it to the diluent-only injections and uninjected eyes, after treating them at 3-4-weeks-old and following them for up to 7 MPI to understand long-term efficacy which was evaluated using multiple experimental modalities, including ERG, OCT, and VGSA.

Our ERG analysis showed although all the doses showed improvement in the cone function, the second highest dose tested, 8E8 vg/eye, had the highest improvements in the retinal function, sustained and restored cone photoreceptor function until 7 MPI treatment compared to the diluent only treatment and the untreated eyes and it was comparable to the WT controls with no statistical significance at earlier and later time points. Our study's OCT-based insights provide a comprehensive grasp of the treatment's impact on retinal structure. Notably, with all vector doses, cyst severity decreased, and there were no observed toxic effects. It's worth emphasizing that while the diluent did contribute to cyst reduction, the vector treatments exhibited better efficacy in mitigating cyst formation at least at the 3 MPI timepoint. This suggests the viral vector's capacity to beneficially reduce cyst development without harming photoreceptor health. The VGSA showed that treated mice with the medium dose had a trend towards improvement in the functional vision in the light and dark, even at older age (9 months) compared to the untreated naïve mice and diluent treated mice. These pieces of evidence support the conclusion that subretinal gene augmentation therapy with the dose 8E8 vg/eye for RS1 significantly restores vision in a Rs1-KO animal model.

The substantial enhancements in both the ERG responses of the rods and cones pathways following subretinal gene therapy point towards a promising functional recovery. The study's findings showcase the remarkable efficacy of the 8E8 vg/eye dose in enhancing cone function, as demonstrated by the 3.0 single flash light-adapted b-wave and the 5 Hz flicker test, with notable improvements observed at 1 MPI, 2 MPI, and 3 MPI compared to the diluent treated and the untreated eyes, and these benefits were impressively sustained for up to 7 MPI compared to the untreated eyes. This suggests the potential of the 8E8 gene therapy to restore the function of off bipolar cells, which play a crucial role in processing rapid changes in light intensity (50).

The reduction of the efficacy of gene therapy in Rs1-KO mice over time, while still surpassing untreated eyes by the study's end, can be attributed to either the deliberate removal of modified cells through immune defense mechanisms, by anti-viral responses (51), or the potential deactivation of therapeutic gene sequences by molecular processes such as episomal silencing (52). While the beneficial effect declines over time, the therapy continues to offer some level of improvement compared to untreated eyes at the study's conclusion.

In a distinct study conducted by our research team, which investigated the buffer tonicity as a treatment to Rs1-KO mice (35), it was observed that mice treated with buffer (whether hypertonic or isotonic) displayed reduced cyst severity and improved cone ERG function in comparison to untreated eyes (Frontiers this issue). Nonetheless, the tonicity of the buffer used played a role in the outcomes. While both hypertonic and isotonic buffer treatments were superior to untreated eyes, it was evident that isotonic buffer-treated mice exhibited a lower effect. This consistency in results aligns with our current findings, particularly considering that the diluent buffer injections in the current study were isotonic. There is evidence by clinical studies that using topical medications like carbonic anhydrase inhibitors (CAIs) in XLRS patients showed reduced cyst severity and better visual acuity (53,54). Oral CAIs can also be effective in improving macular morphology and function in XLRS patients, potentially creating favorable conditions for gene therapy.

Our results in the rod function tests demonstrated that various doses of the AAV2tYF gene therapy vector effectively normalized the hyperactive electrical responses observed in the a-wave amplitudes of the Rs1-KO mouse model. Notably, the 8E8 dose resulted in improved b-wave amplitudes at 1-, 2-, and 3 MPI, indicating enhanced rod photoreceptor function. The b/a ratio after quantifying the SCR test increased progressively in treated eyes due to enhanced b-wave amplitudes, indicating improved signal transmission from photoreceptors to bipolar cells. These findings highlight the treatment potential in preserving visual function.

We have observed that the high (8E9 vg/eye) and low (8E7 vg/eye) gene therapy doses did not significantly improve rod- or cone-specific pathway function. The differing ERG response outcomes of low and high doses compared to the medium dose, can be attributed to factors such as dose-response relationships, potential saturation effects at high doses, the complexity of the disease and its response to treatment, as well as the potential dose-dependent behavior of certain factors. The optimal improvements seen with the medium dose might stem from a balanced interaction of these factors, resulting in the observed favorable ERG enhancements.

The improvements we observed by the medium 8E8 dose were strengthened by the results of the VGSA, indicating a trend toward improved visual function. This alignment between electrophysiological measures and functional outcomes underscores the potential clinical relevance of our findings. The presence of high recordings in the ERG signal does not directly correlate with the presence of functional vision. The most direct measure of therapeutic efficacy is functional vision. Conducted at 4-5 months of age and then again at 9 months age, these experiments showed that the 8E8 vg/eye gene therapy dosage had a trend towards improvement in functional vision in these mice. 8E8 treated Rs1-KO mice took 20.3% and 28.4% less time than untreated Rs1-KO mice to complete visual tasks in the light and in the dark respectively at 9 months of age.

Our findings emphasize the dose-dependent nature of gene augmentation's efficacy in preserving rod photoreceptors and restoring cone electrical function. Thus, the dosage of gene therapy plays a pivotal role in determining its effectiveness in mitigating retinal degeneration in this context. These insights contribute to the understanding of gene therapy as a potential treatment strategy for XLRS.

### Bridging Bench to Bedside: Translating Subretinal Gene Therapy to Clinical Practice

The translational implications of our study are twofold. First, the encouraging outcomes observed in our murine model lay a strong foundation for further preclinical investigations, in which the limits of durability can be studied. Second, our findings pave the way for potential clinical trials in humans, suggesting the subretinal route of administration in selected patients may offer better efficacy.

### REFERENCES

1. C. Vijayasarathy, Y. Zeng, M. J. Brooks, R. N. Fariss, P. A. Sieving, Genetic Rescue of X-Linked Retinoschisis Mouse (Rs1(−/y)) Retina Induces Quiescence of the Retinal Microglial Inflammatory State Following AAV8-RS1 Gene Transfer and Identifies Gene Networks Underlying Retinal Recovery. Hum Gene Ther 32, 667-681 (2021).
2. A. E. Gehrig, R. Warneke-Wittstock, C. G. Sauer, B. H. Weber, Isolation and characterization of the murine X-linked juvenile retinoschisis (Rs1h) gene. Mamm Genome 10, 303-307 (1999).
3. C. G. Sauer et al., Positional cloning of the gene associated with X-linked juvenile retinoschisis. Nat Genet 17, 164-170 (1997).
4. R. S. Molday, U. Kellner, B. H. Weber, X-linked juvenile retinoschisis: clinical diagnosis, genetic analysis, and molecular mechanisms. Prog Retin Eye Res 31, 195-212 (2012).
5. G. J. Ye et al., Safety and Biodistribution Evaluation in Cynomolgus Macaques of rAAV2tYF-CB-hRS1, a Recombinant Adeno-Associated Virus Vector Expressing Retinoschisin. Hum Gene Ther Clin Dev 26, 165-176 (2015).
6. Strupaité R, Ambrozaityté L, Cimbalistiené L, Ašoklis R, Utkus A. X-linked juvenile retinoschisis: phenotypic and genetic characterization. Int J Ophthalmol. 2018 Nov. 18; 11(11):1875-1878. doi: 10.18240/ijo.2018.11.22. PMID: 30450322; PMCID: PMC6232335.
7. Murro V, Caputo R, Bacci G M, Sodi A, Mucciolo D P, Bargiacchi S, Giglio S R, Virgili G, Rizzo S. Case report of an atypical early onset X-linked retinoschisis in monozygotic twins. BMC Ophthalmol. 2017 Feb. 24; 17(1):19. doi: 10.1186/s12886-017-0406-6. PMID: 28235399; PMCID: PMC5324242.
8. A. Tantri et al., X-linked retinoschisis: a clinical and molecular genetic review. Surv Ophthalmol 49, 214-230 (2004).
9. N. D. George, J. R. Yates, A. T. Moore, X linked retinoschisis. Br J Ophthalmol 79, 697-702 (1995).
10. Testa F, Di Iorio V, Gallo B, et al. Carbonic anhydrase inhibitors in patients with X-linked retinoschisis: effects on macular morphology and function. Ophthalmic Genet. 2019; 40:207e212.
11. Min S H, Molday L L, Seeliger M W, et al. Prolonged recovery of retinal structure/function after gene therapy in an Rs1hdeficient mouse model of x-linked juvenile retinoschisis. Mol Ther. 2005; 12:644e651.
12. Kjellstrom S, Bush R A, Zeng Y, et al. Retinoschisin gene therapy and natural history in the Rs1h-KO mouse: long-term rescue from retinal degeneration. Invest Ophthalmol Vis Sci. 2007; 48, 3837e3345.
13. Byrne L C, Ozturk B E, Lee T, et al. Retinoschisin gene therapy in photoreceptors, Muller glia or all retinal cells in the Rs1h−/− mouse. Gene Ther. 2014; 21:585e592.
14. Y. Zeng et al., RS-1 Gene Delivery to an Adult Rs1h Knockout Mouse Model Restores ERG b-Wave with Reversal of the Electronegative Waveform of X-Linked Retinoschisis. Invest Ophthalmol Vis Sci 45, 3279-3285 (2004).
15. A. Janssen et al., Effect of late-stage therapy on disease progression in AAV-mediated rescue of photoreceptor cells in the retinoschisin-deficient mouse. Mol Ther 16, 1010-1017 (2008).
16. Y. Takada et al., Synaptic pathology in retinoschisis knockout (Rs1−/y) mouse retina and modification by rAAV-Rs1 gene delivery. Invest Ophthalmol Vis Sci 49, 3677-3686 (2008).
17. T. K. Park et al., Intravitreal delivery of AAV8 retinoschisin results in cell type-specific gene expression and retinal rescue in the Rs1-KO mouse. Gene Ther 16, 916-926 (2009).
18. Cukras C, Wiley H E, Jeffrey B G, et, al, Retinal AAV8-RS1 Gene Therapy for X-Linked Retinoschisis: Initial Findings from a Phase Ulla Trial by Intravitreal Delivery. Mol Ther. 2018; 26(9):2282-2294, doi:10.1016/j.ymthe.2018.05.025
19. Pennesi M E, Yang P, Birch D G, et al. Intravitreal Delivery of rAAV2tYF-CB-hRS1 Vector for Gene Augmentation Therapy in Patients with X-Linked Retinoschisis: 1-Year Clinical Results. Ophthalmol Retina. 2022; 6(12)
20. Wu, Z, Asokan, A and Samulski, R J (2006). Adeno-associated virus serotypes: vector toolkit for human gene therapy. Mol Ther 14: 316-327.
21. Zhong, L, Li, B, Jayandharan, G, Mah, C S, Govindasamy, L, Agbandje-McKenna, M et al. (2008). Tyrosine-phosphorylation of AAV2 vectors and its consequences on viral intracellular trafficking and transgene expression. Virology 381: 194-202.
22. Zhong, L, Li, B, Mah, CS, Govindasamy, L, Agbandje-McKenna, M, Cooper, M et al. (2008). Next generation of adeno-associated virus 2 vectors: point mutations in tyrosines lead to high-efficiency transduction at lower doses. Proc Natl Acad Sci USA 105: 7827-7832.
23. Li Q, Miller R, Han P Y, et al. Intraocular route of AAV2 vector administration defines humoral immune response and therapeutic potential. Mol Vis. 2008; 14: 1760-1769.
24. Kotterman M A, Yin L, Strazzeri J M, Flannery J G, Merigan W H, Schaffer D V. Antibody neutralization poses a barrier to intravitreal adeno-associated viral vector gene delivery to non-human primates. Gene Ther. 2015; 22: 116-126.
25. Seo S, Mullins R F, Dumitrescu A V, Bhattarai S, Gratie D, Wang K, Stone E M, Sheffield V, Drack A V. Subretinal gene therapy of mice with Bardet-Biedl syndrome type 1. Invest Ophthalmol Vis Sci. 2013 Sep. 11; 54(9):6118-32. doi: 10.1167/iovs.13-11673. PMID: 23900607; PMCID: PMC3771708.
26. Scruggs B A, Bhattarai S, Helms M, Cherascu I, Salesevic A, Stalter E, Laird J, Baker S A, Drack A V. AAV2/4-RS1 gene therapy in the retinoschisin knockout mouse model of X-linked retinoschisis. PLoS One. 2022 Dec. 7; 17(12):e0276298. doi: 10.1371/journal.pone.0276298. PMID: 36477475; PMCID: PMC9728878.
27. Hsu Y, Bhattarai S, Thompson J M, Mahoney A, Thomas J, Mayer S K, Datta P, Garrison J, Searby C C, Vandenberghe L H, Seo S, Sheffield V C, Drack A V. Subretinal gene therapy delays vision loss in a Bardet-Biedl Syndrome type 10 mouse model. Mol Ther Nucleic Acids. 2022 Dec. 12; 31:164-181. doi: 10.1016/j.omtn.2022.12.007. PMID: 36700052; PMCID: PMC9841241.
28. Robson A G, Frishman L J, Grigg J, Hamilton R, Jeffrey B G, Kondo M, Li S, McCulloch D L. ISCEV Standard for full-field clinical electroretinography (2022 update). Doc Ophthalmol. 2022 June; 144(3):165-177. doi: 10.1007/s10633-022-09872-0. Epub 2022 May 5. PMID: 35511377; PMCID: PMC9192408.
29. Jagodzinska J, Sarzi E, Cavalier M, et al. Optical Coherence Tomography: Imaging Mouse Retinal Ganglion Cells In Vivo. J Vis Exp. 2017; (127):55865. Published 2017 Sep. 22. doi:10.3791/55865
30. Bush R A, Zeng Y, Colosi P, Kjellstrom S, Hiriyanna S, Vijayasarathy C, Santos M, Li J, Wu Z, Sieving P A. Preclinical Dose-Escalation Study of Intravitreal AAV-RS1 Gene Therapy in a Mouse Model of X-linked Retinoschisis: Dose-Dependent Expression and Improved Retinal Structure and Function. Hum Gene Ther. 2016 May; 27(5):376-89. doi: 10.1089/hum.2015.142. PMID: 27036983; PMCID: PMC4840830.
31. Hassan S, Hsu Y, Mayer S K, Thomas J, Kothapalli A, Helms M, et al. A visually guided swim assay for mouse models of human retinal disease recapitulates the multi-luminance mobility test in humans. Saudi J Ophthalmol 2023 (in press)
32. Morris R. Developments of a water-maze procedure for studying spatial learning in the rat. J Neurosci Methods 1984; 11:47-60.
33. Pang J J, Chang B, Kumar A, Nusinowitz S, Noorwez S M, Li J, et al. Gene therapy restores vision-dependent behavior as well as retinal structure and function in a mouse model of RPE65 leber congenital amaurosis. Mol Ther 2006; 13:565-72.
34. Renner A B, Kellner U, Fiebig B, Cropp E, Foerster M H, Weber B H. ERG variability in X-linked congenital retinoschisis patients with mutations in the RS1 gene and the diagnostic importance of fundus autofluorescence and OCT. Doc Ophthalmol. 2008; 116(2):97-109. doi:10.1007/s10633-007-9094-5
35. Jacob Thompson; Ying Hsu; Ella Gehrke; Salma Hassan; Sara Kathryn Mayer; Angela Mahoney; Arlene V Drack. Resolution of retinal cysts following subretinal buffer injection in a mouse model of Juvenile X-linked Retinoschisis, ARVO Annual Meeting Abstract June 2023
36. Breton M E, Schueller A W, Lamb T D, Pugh E N Jr. Analysis of ERG a-wave amplification and kinetics in terms of the G-protein cascade of phototransduction. Invest Ophthalmol Vis Sci. 1994:35(1):295-309.
37. Penn R D, Hagins W A. Signal transmission along retinal rods and the origin of the electroretinographic a-wave. Nature. 1969; 223(5202):201-204, doi:10.1038/223201a0
38. Newman E A, Odette L L. Model of electroretinogram b-wave generation: a test of the K+ hypothesis. Neurophysiol. 1984; 51(1):164-182. doi:10.1152/jn.1984.51.1.164
39. Robson J G, Erishman L J. Response linearity and kinetics of the cat retina: the bipolar cell component of the dark-adapted electroretinogram. Vis Neurosci. 1995; 12(5):837-850. doi:10.1.017/s0952523800009408
40. Stockton R A, Slaughter M M. B-wave of the electroretinogram. A reflection of (bipolar cell activity. J Gen Physiol. 1989; 93(1):101-122. doi:10.10851jgp.93.1.101
41. Xu X, Karwoski C J. Current source density analysis of retinal field potentials. IL Pharmacological analysis of the b-wave and M-wave. J. Neurophysiol. 1994; 72(1):96-105. doi:10.1152/jn.1994.72.1.96
42. Zeng Y, Petralia R S, Vijayasarathy C, et al. Retinal Structure and Gene Therapy Outcome in Retinoschisin-Deficient Mice Assessed by Spectral-Domain Optical Coherence Tomography. Invest Ophthalmol Vis Sci. 2016; 57(9):OCT277-OCT287. doi:10.1167/iovs.15-18920
43. Russell S, Bennett J, Wellman J A, et al. Efficacy and safety of voretigene neparvovec (AAV2-hRPE65v2) in patients with RPE65-mediated inherited retinal dystrophy: a. randomised, controlled, open-label, phase 3 trial [published correction appears in Lancet, 2017 Aug. 26; 390(10097):848]. Lancet. 2017; 390(10097):849-860. doi:10.10.16/S0140-6736(17)31868-8
44. Chung D C, McCague S, Yu Z F, et al. Novel mobility test to assess functional vision in patients with inherited retinal dystrophies. Clin Exp Ophthalmol, 2018; 46(3):247-259. doi:10.11111/ceo.13022
45. Mayer S K, Thomas J, Helms M, et al. Progressive retinal degeneration of rods and cones in a Bardet-Biedl syndrome type 10 mouse model. Dis Model Mech. 2022; 15(9):dmm049473. doi:10.1242/dmm.049473
46. Bucher K, Rodríguez-Bocanegra E, Dauletbekov D, Fischer M D. Immune responses to retinal gene therapy using adeno-associated viral vectors—Implications for treatment success and safety. Prog Retin Eye Res. 2021; 83:100915. doi:10.1016/j.preteyeres.2020.100915
47. Timmers A M, Newmark J A, Turunen H T, et al. Ocular Inflammatory Response to Intravitreal Injection of Adeno-Associated Virus Vector: Relative Contribution of Genome and Capsid. Hum Gene Ther. 2020; 31(1-2):80-89. doi:10.11.089/hum.2019.144
48. Jiang X, Mahroo O A. Negative electroretinograms: genetic and acquired causes, diagnostic approaches and physiological insights. Eye (Land). 2021 September; 35(9):2419-2437. doi: 10.1038/s41433-021-01604-z. Epub 2021 Jun. 14. PMID: 34127841; PMCID: PMC8377097.
49. Chatterjee S, Gupta. S, Kirola L, Chandra A, Mukheijee A, Mutsuddi M, identification and molecular characterization of two recurrent missense mutations in RS1 gene in two families with X-linked retinoschisis from North India [published online ahead of print, 2023 Jun. 15]. Am J Med Genet A. 2023; 10.1002/ajmg.a.63327. doi:10.1002/ajm.g.a.63327
50. Tanimoto N, Sothilingam V, Kondo M, Biel M, Humphries P, Seeliger M W. Electroretinographic assessment of rod- and cone-mediated bipolar cell pathways using flicker stimuli in mice. Sci Rep. 2015 Jun. 1; 5:10731. doi: 10.1038/srep10731. PMID: 26029863; PMCID: PMC5377071.
51. Bucher K, Rodríguez-Bocanegra E, Dauletbekov D, Fischer M D. Immune responses to retinal gene therapy using adeno-associated viral vectors—Implications for treatment success and safety. Prog Retin Eye Res. 2021 July; 83:100915. doi: 10.1016/j.preteyeres.2020.100915. Epub 2020 Oct. 15. PMID: 33069860.
52. Gray S J, Foti S B, Schwartz J W, Bachaboina L, Taylor-Blake B, Coleman J, Ehlers M D, Zylka M J, McCown T J, Samulski R J. Optimizing promoters for recombinant adeno-associated virus-mediated gene expression in the peripheral and central nervous system using self-complementary vectors. Hum Gene Ther. 2011 September; 22(9):1143-53. doi: 10.1089/hum.2010.245. Epub 2011 Jun. 1. PMID: 21476867; PMCID: PMC3177952.
53. Testa F, Di Iorio V, Gallo B, et al. Carbonic anhydrase inhibitors in patients with X-linked retinoschisis: effects on macular morphology and function. Ophthalmic Genet, 2019; 4013):207-212. doi:10.1.080/1381681.0.201.9.1616303
54. Apushkin M A, Fishman G A. Use of dorzolamide for patients with X-linked retinoschisis [published correction appears in Retina. 2007 January; 27(1):1281. Retina. 2006:26(7):741-745. doi:10.1.097/01.iae.0000237081.80600.51.

The present invention is not to be limited in scope by the specific embodiments described herein. It will be appreciated that the invention is susceptible to modification, variation and change without departing from the spirit thereof.

What is claimed is:

1. A method of treating X-linked juvenile retinoschisis (XLRS) in a human subject, comprising:
subretinally delivering to the human subject a therapeutically effective amount of an rAAV vector, the rAAV vector comprising a nucleic acid sequence comprising coding sequence for human RS1 protein.

2. The method of claim 1, wherein the rAAV vector further comprises a mutated AAV2 VP3 capsid protein comprising phenylalanine (F) for tyrosine (Y) substitutions at each of the positions corresponding to Y444, Y500 and Y730 in a wild type AAV2 VP3 capsid protein.

3. The method of claim 1 or 2, wherein the nucleic acid sequence of the rAAV vector further comprises a chicken beta actin promoter sequence.

4. The method of claim 1 or 2, wherein the nucleic acid sequence of the rAAV vector further comprises a CMV enhancer.

5. The method of any of the foregoing claims, wherein the rAAV vector is rAAV2tYF-CB-hRS1.

6. A method of treating X-linked juvenile retinoschisis (XLRS) in a human subject, comprising:
subretinally delivering to the human subject a pharmaceutical composition comprising rAAV2tYF-CB-hRS1 and a pharmaceutically-acceptable carrier.
